(19) United States
(12) Patent Application Publication
(10) Pub. No.: US 2025/0242139 A1
(43) Pub. Date: Jul. 31, 2025

(54) CATHETER ASSEMBLY

(71) Applicant: ConvaTec Limited, Flintshire (GB)

(72) Inventors: Patrik Lacko, Flintshire (GB); Marian Novak, Flintshire (GB)

(21) Appl. No.: 19/183,013

(22) Filed: Apr. 18, 2025

Related U.S. Application Data
(63) Continuation of application No. PCT/GB2023/052693, filed on Oct. 17, 2023.
(60) Provisional application No. 63/417,497, filed on Oct. 19, 2022.
(60) Provisional application No. 63/417,506, filed on Oct. 19, 2022.
(60) Provisional application No. 63/417,511, filed on Oct. 19, 2022.

(30) Foreign Application Priority Data
Nov. 10, 2022 (GB) 2216801.7
Nov. 10, 2022 (GB) 2216803.3
Nov. 10, 2022 (GB) 2216804.1

(51) Int. Cl. A61M 27/00; A61M 39/24

(52) U.S. Cl. CPC A61M 27/002; A61M 39/24; A61M 2039/2426; A61M 2202/0496; A61M 2207/00

(57) ABSTRACT

A catheter assembly comprises a catheter and a wetting mechanism. The catheter has a proximal end for insertion into the body and a distal end. The wetting mechanism is arranged at the proximal end and comprises a body. The body defines a wetting chamber through which the catheter may be moved to wet the catheter. The wetting mechanism may comprise a slit valve or sealing element configured to allow passage of the catheter into the wetting chamber and to inhibit release of fluid from the wetting chamber. The catheter may be an intermittent male urinary catheter, and may be 10 hydrophilic. The catheter assembly may comprise two slit valves. The sealing element may be compressed by the wetting mechanism. The sealing element may be abutted by the wetting mechanism to support it in the transverse and axial directions. The sealing element may have a stepped profile.

### TECHNICAL FIELD OF THE INVENTION

The present invention relates to catheter assemblies. In particular the invention concerns urinary catheter assemblies, and most particularly, but not exclusively intermittent urinary catheter assemblies.

### BACKGROUND TO THE INVENTION

A catheter is a medical device comprising a hollow catheter tube designed for insertion into canals, vessels, passageways or body cavities to permit injection, drainage or withdrawal of fluids or substances therefrom, or to ensure said canals, vessels, passageways etc. remain open. Urinary catheters are designed for use for insertion into a user's bladder via the urethra to drain the bladder.

To maximise comfort and minimise the risk of trauma and/or infection, an outer surface of the catheter tube is typically wetted using a wetting agent prior to insertion by the user. In further developments, the catheter tube itself comprises, is integrated with or is coated with a hydrophilic component (e.g. a hydrophilic polymer) which serves to reduce friction further upon application of the wetting agent.

Some catheters may be supplied pre-wetted in a packaging, for instance, where the catheter is at least partially submerged within wetting agent within the packaging. Whilst this may ensure the catheter tube is adequately wetted prior to use, such arrangements suffer in that components of the catheter other than the catheter tube such as a gripper element or funnel can also become wetted. This has a detrimental effect of the experience of the user where it may become difficult to hold and direct the catheter tube as required. This is particularly problematic where the user is performing self-catheterisation. Further, having the catheter submerged may effectively reduce the shelf-life of the catheter due to long-term exposure of components of the catheter to moisture.

It is therefore seen as advantageous to provide a catheter which may be wetted at or immediately prior to the point of use.

In an attempt to address this, some catheters are provided in packaging which includes a rupturable container or sachet within the packaging which a user may burst to release the wetting agent. Typically, this involves the user squeezing the packaging to cause the container/sachet to break. However, such arrangements experience similar problems to those discussed above where the wetting agent is allowed to come into contact with other components of the catheter. Such arrangements also result in the possibility of the catheter tube not being fully wetted, or indeed wetted at all, prior to use. This can be harmful for the user. Furthermore such systems may require a degree of dexterity and offer no feedback to ensure wetting has occurred.

It is therefore advantageous to provide a catheter which includes a means of easily supplying a wetting agent solely to the catheter tube to improve user experience.

Manual dexterity can also be a problem when opening packaging in order to access the catheter as it can lead the user to incorrectly opening packaging, for example by tearing, which can result in the catheter coming into contact with dirt and being rendered unsafe for use. It is therefore advantageous to provide a catheter in packaging that is may be easily opened by the user while maintaining the catheter in a clean and usable state.

It is an aim of an embodiment or embodiments of the invention to overcome or at least partially mitigate one or more problems with the prior art and/or to provide an improved intermittent catheter.

### SUMMARY OF THE INVENTION

The invention concerns a catheter assembly. The assembly may comprise a catheter comprising a proximal end for insertion into the user and a distal end, and a wetting mechanism. The wetting mechanism may comprise a body. The wetting mechanism may comprise a wetting chamber. The body may define the wetting chamber through which the catheter may be moved to wet the catheter. The wetting mechanism may comprise a valve. The valve may be configured to inhibit release of fluid from the wetting chamber. The valve may be configured to allow passage of the catheter into the wetting chamber and consequently may be an inlet valve. The valve is preferably a slit valve, for example the inlet valve is preferably a slit valve.

The wetting mechanism may comprise a sealing element. The sealing element may comprise the valve, for example the inlet valve. The valve may define a transverse plane perpendicular to the axial direction. The sealing element may have a stepped profile. The sealing element may comprise an outer wall extending parallel to the transverse plane but offset from the valve. The sealing element may comprise an inner support extending in the axial direction. The inner support may connect the outer wall to the valve. The wetting mechanism may abut the inner support to inhibit movement of the valve in the transverse plane. The wetting mechanism may abut the outer wall to inhibit movement of the valve in the axial direction.

The sealing element may comprise a top-hat profile. The top-hat profile may be provided by the valve, inner support and outer wall. The valve may be provided in the centre of the top-hat profile. Thus, the sealing element may be better supported due to the different parts of the top-hat profile that can be abutted by the rest of the wetting mechanism.

The wetting mechanism may comprise a base. The wetting chamber may be tubular. The body may comprise an opening. The opening may be sized to allow the catheter to pass through the base. The opening may be sized to allow the catheter to pass into the wetting chamber. The sealing element may comprise a material that is more flexible than the body and/or the base. The sealing element may be resiliently deformed by the body and base, preferably to provide a fluid-tight seal between the body and the base.

According to a broad aspect of the invention there is provided a catheter assembly comprising: a catheter comprising a proximal end for insertion into the user and a distal end, and a wetting mechanism wherein the wetting mechanism comprises a body, the body defines a wetting chamber through which the catheter may be moved to wet the catheter, and wetting mechanism comprises a slit valve configured to allow passage of the catheter into the wetting chamber and inhibit release of fluid from the wetting chamber.

The wetting mechanism may be arranged at the proximal end of the catheter.

According to a first aspect of the invention there is provided a catheter assembly comprising: a catheter comprising a proximal end for insertion into the user and a distal end, and a wetting mechanism arranged at the proximal end of the catheter, wherein the wetting mechanism comprises a body, the body defines a wetting chamber through which the catheter may be moved to wet the catheter, and wetting mechanism comprises a slit valve configured to allow passage of the catheter into the wetting chamber and inhibit release of fluid from the wetting chamber.

Advantageously, as the catheter can pass through the wetting mechanism it can come into direct contact with wetting fluid and therefore be efficiently wetted by it. In addition, the use of slit valve ensures the wetting mechanism is simple and cheap to construct while providing a robust and easy to use valve as the catheter can be simply pushed through the valve and into the wetting chamber. Furthermore, as the wetting mechanism is arranged at the proximal end of the catheter, this helps ensure the proximal end of the catheter is adequately wetted which is important given it is the first part of the catheter to enter the body in use. The catheter assembly of the first aspect is therefore easy to use, reduces risk of injury/discomfort to the user and is also easy to manufacture.

The slit valve may comprise at least one slit. The slit valve may comprise at least two slits, and preferably the slit valve comprises only two slits. Each slit may have a length that is greater than the diameter of the catheter. Each slit may have a length that is no more than 20%, 30%, 40%, 50%, 60%, 70% or 80% a diameter of the slit valve. Each slit may have a length that is no less than 80%, 70%, 60%, 50%, 40%, 30% or 20% the diameter of the slit valve. Preferably, each slit has a length of 70% the diameter of the slit valve, for example 5-6 mm or most preferably 5.5 mm. Each slit may have substantially the same length. Thus, the slits can provide an effective seal without negatively effecting the structural integrity of the slit valve.

One or more or each slit may be arranged to intersect a centre of the slit valve. one or more or each slit may extend from the centre of the slit valve. One or more or each slit may terminate at substantially the same distance from the centre of the slit valve. Where there are two or more slits, at least two slits, or each slit, may intersect. Where there are two or more slits, there may be two or more angles defined between adjacent slits. The two or more angles may all be substantially the same. The two slits (or at least two of the slits) may be arranged orthogonally. The two slits (or at least two of the slits) may be arranged in a cross. The cross may be centred on the centre of the slit valve. Thus, the slits may be arranged to maximise the available space and ensure that the catheter passes through the middle of the slit valve and into the wetting chamber efficiently.

In some embodiments, the two or more angles between adjacent slits may be different, that is they may not be equal. For example, in one embodiment, two slits may be arranged in a T-shape to define three flaps. In another embodiment three slits may be arranged in a Y-shape to define three flaps.

The slit valve may comprise at least two flaps. The at least one slit may define the at least two flaps. The at least two flaps may be movable to allow the valve to be opened. The at least two flaps may be separatable. The at least two flaps may be movable/separatable by the catheter to allow passage of the catheter through the slit valve. The slit valve may comprise four flaps preferably defined by two slits. Thus, the slit valve can be easily opened and closed via the flaps.

An axial direction may be defined as the direction in which the catheter passes into the wetting chamber through the slit valve. A transverse plane may be defined by the slit valve perpendicular to the axial direction.

The slit valve may be any suitable shape or size, for example circular, elliptical, lens-shaped, triangular, rectangular, square, or irregularly shaped. Preferably the slit valve is circular. The slit valve may have a width, for example a diameter, that is larger than the diameter of the catheter, for example at least 2 times, 3 times, 4 times, or 5times larger than the diameter of the catheter. The slit valve may have a width of at least 5 mm, 10 mm or 15 mm. The slit valve may have a width of no more than 15 mm, 10 mm or 5 mm. Preferably, the width of the slit valve is 5-10 mm and most preferably about 8 mm. Thus, the valve is easily and flexibly incorporated into various wetting mechanism designs.

The slit valve may be formed of a flexible resilient material such as a flexible plastics material, rubber or silicone. The slit valve may be resiliently deformable. The slit valve may be normally closed. Each flap may be resiliently biased to return to a closed position. In the closed position, the two or more flaps may co-operate to seal the slit valve. Thus, the slit valve can be easily constructed to automatically seal the wetting chamber and prevent leaks of fluid without use of complex parts.

The slit valve may have a curved surface. The slit valve may be domed. The slit valve may have a dome height parallel to the axial direction. The dome height may be at least 1%, 2%, 5%, 10%, 15% or 20% the diameter of the slit valve. The dome height may be no more than 30%, 20%, 15%, 10%, 5%, 2% or 1% the diameter of the slit valve. Preferably, the dome height is 10% the diameter of the slit valve, for example about 1 mm. The slit valve may be concave on a side facing the wetting chamber. The slit valve may be convex on a side facing away from the wetting chamber. Thus, the slit valves are able to more effectively prevent leaks of wetting fluid from the wetting chamber.

The wetting mechanism may comprise a sealing element which may comprise an inlet valve which may be the slit valve. The inlet valve may define a transverse plane perpendicular to the axial direction. The sealing element may have a stepped profile. The sealing element may comprise an outer wall extending parallel to the transverse plane but offset from the inlet valve. The sealing element may comprise an inner support extending in the axial direction. The inner support may connect the outer wall to the inlet valve. The wetting mechanism may abut the inner support to inhibit movement of the inlet valve in the transverse plane. The wetting mechanism may abut the outer wall to inhibit movement of the inlet valve in the axial direction.

According to another broad aspect of the invention there is provided a catheter assembly comprising: a catheter comprising a proximal end for insertion into the body and a distal end, and a wetting mechanism wherein the wetting mechanism comprises a wetting chamber and a sealing element, the sealing element comprising an inlet valve configured to allow passage of the catheter into the wetting chamber in an axial direction to be wetted by wetting fluid, the inlet valve is configured to inhibit release of fluid from the wetting chamber, the inlet valve defines a transverse plane perpendicular to the axial direction, the sealing element comprises: a stepped profile, an outer wall extending parallel to the transverse plane but offset from the inlet valve, an inner support extending in the axial direction and connecting the outer wall to the inlet valve, wherein the wetting mechanism abuts the inner support to inhibit movement of the inlet valve in the transverse plane, and the wetting mechanism abuts the outer wall to inhibit movement of the inlet valve in the axial direction.

The wetting mechanism may be arranged at the proximal end of the catheter.

According to a second aspect of the invention there is provided a catheter assembly comprising: a catheter comprising a proximal end for insertion into the body and a distal end, and a wetting mechanism arranged at the proximal end of the catheter wherein the wetting mechanism comprises a wetting chamber and a sealing element, the sealing element comprising an inlet valve configured to allow passage of the catheter into the wetting chamber in an axial direction to be wetted by wetting fluid, the inlet valve is configured to inhibit release of fluid from the wetting chamber, the inlet valve defines a transverse plane perpendicular to the axial direction, the sealing element comprises: a stepped profile, an outer wall extending parallel to the transverse plane but offset from the inlet valve, an inner support extending in the axial direction and connecting the outer wall to the inlet valve, wherein the wetting mechanism abuts the inner support to inhibit movement of the inlet valve in the transverse plane, and the wetting mechanism abuts the outer wall to inhibit movement of the inlet valve in the axial direction.

Advantageously the inlet valve is supported via the inner support and outer wall and which minimises obstruction of the inlet valve by the rest of the wetting mechanism. The inlet valve can therefore be most efficiently sized for allowing the passage of the catheter therethrough. In addition, this arrangement allows the outer wall and inner support to be designed in a way that maximises the structural support of the inlet valve so the inlet valve can perform more effectively. Furthermore, as the wetting mechanism is arranged at the proximal end of the catheter, this helps ensure the proximal end of the catheter is adequately wetted which is important given it is the first part of the catheter to enter the body in use.

The sealing element may be any suitable shape or size, such as circular, elliptical, lens-shaped, triangular, rectangular, square, or irregularly shaped, preferably the sealing element is circular. The sealing element may be sheet-like, for example it may have a substantially constant thickness.

The inner support may surround at least a part of the inlet valve. The inner support may surround a majority of the inlet valve, preferably in the transverse plane. The inner support may be continuous. Thus, the inner support can be used to surround the inlet valve which ensures that is can provide good structural support in both the transverse plane but also the axial direction without necessarily getting in the way of the catheter passing through the inlet valve.

The inner support may be attached to an outer edge of the inlet valve. The inlet valve may be curved towards the inner support. The inlet valve may be concave on a side corresponding to the inner support. Thus, the inner support provides support via the edges of the valve and doesn't obstruct the valve's operation.

The inner support may comprise an inner flange. The inner flange may extend in the axial direction. The inner flange may surround the inlet valve in the transverse plane. The inner flange may be frustoconical. A narrow end of the inner flange may be attached to the inlet valve, preferably the outer edge of the inlet valve. A wide end of the inner flange may be at least 1%, 2%, 5%, or 10% larger in diameter than the narrow end. The wide end of the inner flange may be no more than 10%, 5%, 2%, or 1% larger in diameter than the narrow end. Preferably, the wide end of the inner flange has a diameter 5% larger than the narrow end. In one example, the narrow end of the inner flange has a diameter of 8 mm and the wide end a diameter of 8.5 mm. The inner flange may have a length between the narrow end and the wide end measured in the axial direction of at least 15%, 20%, 25% or 30% the diameter of the inlet valve. The inner flange may have a length of no more than 30%, 25%, 20% or 15% the diameter of the inlet valve. Preferably the inner flange has a length of 2% the diameter of the inlet valve. In one example, the inner flange has a length of about 2 mm. Thus, the inner flange provides a surface that extends in the axial direction as well as around the inlet valve. This can be abutted by the wetting mechanism to effectively inhibit movement of the inlet valve in the transverse plane from a position outside the transverse extent of the inlet valve. Therefore, the inlet valve itself is not obstructed by the wetting mechanism.

The wetting mechanism may comprise a transverse support structure configured to abut the inner support. The transverse support structure may be configured to inhibit movement of the inner support and inlet valve in the transverse plane. The transverse support structure may surround at least part of the inner support. The transverse support structure may surround the inner support, preferably in the transverse plane. The transverse support structure may comprise an inner sealing tube. The inner sealing tube may be configured to abut the inner flange. The inner sealing tube may be cylindrical. The inner sealing tube may have an inner diameter that is less than the diameter of the wide end of the inner flange. The inner sealing tube may have an inner diameter that is greater than the diameter of the narrow end of the inner flange. The inner sealing tube may be configured to resiliently deform the sealing element/inner flange. The inner sealing tube may have a length that is longer than the inner flange. Thus, the transverse support structure/inner sealing tube can effectively surround the inner flange and inlet valve to inhibit their movement in the transverse plane, as the inner flange can be wedged inside the inner sealing tube. Of course, in other embodiments a variation on the transverse support structure/inner sealing tube could be used to achieve the same effect, for example a tube is not always necessary and could be replaced for example with a series of support members, e.g. bars, arranged around the inner support/inner flange.

The outer wall may surround at least a part of the inlet valve and/or inner support. The outer wall may surround the inlet valve and/or inner support, preferably in the transverse plane. The outer wall may be continuous. The outer wall may be annular. The outer wall may comprise an inner perimeter corresponding to an outer perimeter of the inlet valve. The inner perimeter of the outer wall may be spaced outside the outer perimeter of the inlet valve in the transverse direction. The outer wall may have an inner diameter that is larger than the diameter of the inlet valve. The outer wall may have an inner diameter equivalent to the diameter of the wide end of the inner flange. The outer wall may have an outer diameter that is at least 50%, 60%, 65%, 70% or 80% larger than the diameter of the inlet valve. The outer wall may have an outer diameter that is no more than 80%, 70%, 65%, 70% or 50% larger than the diameter of the inlet valve. Preferably, the outer diameter of the outer wall is about 70% larger than the diameter of the inlet valve, for example about 14 mm. The outer wall may be offset from the inlet valve in the transverse plane by the length of the inner support in the axial direction. Thus, the outer wall can be used to surround both the inner support and inlet valve which ensures that is can provide good structural support in both the transverse plane but also the axial direction without necessarily getting in the way of the catheter passing through the inlet valve or the inner support in supporting the inlet valve.

The sealing element may comprise an outer support. The outer support may surround at least part of the outer wall. The outer support may surround a majority of the outer wall, preferably in the transverse plane. The outer support may be continuous. The outer support may be attached to the outer wall, preferably at the outer edge of the outer wall. The outer support may extend away from the inner support in the axial direction. Thus, the outer support can be used to surround the outer wall which ensures that is can provide good structural support in both the transverse plane but also the axial direction without necessarily getting in the way of the catheter passing through the inlet valve.

The outer support may be attached to an outer edge of the outer wall. Thus, the outer support provides support via the edges of the outer wall and doesn't obstruct the inlet valve's operation.

The wetting mechanism may comprise an axial support structure. The axial support structure may be configured to abut the outer wall and/or inner support. The axial support structure may be configured to inhibit movement of the outer wall, inner support and/or inlet valve in the axial direction. The axial support structure may be arranged on one, or preferably both, side(s) of the outer wall in the axial direction. The axial support structure may comprise the inner sealing tube. The inner sealing tube may be configured to abut the outer wall, preferably around the inner diameter of the outer wall adjacent the inner flange. The inner sealing tube may be configured to prevent over insertion of the inlet valve into the inner sealing tube. Thus, the axial support structure/inner sealing tube can effectively inhibit movement of the inlet valve in the axial direction as the outer wall abuts the inner sealing tube. This helps to ensure the catheter can open the valve and move through it, as well as preventing damage to the inlet valve/inner flange due to over insertion into the inner sealing tube.

The axial support structure may be configured to abut the outer wall at more than one location, for example at two or more independent locations. This can help to ensure the outer wall does not excessively bend or deform whilst being supported.

The axial support structure may comprise an outer sealing tube. The outer sealing tube may be arranged co-axially with the inner sealing tube. The outer sealing tube may be configured to abut the outer wall, preferably on the same side of the outer wall as the inner sealing tube. The outer sealing tube may have an inner diameter that is less than the outer diameter of the outer wall. The outer sealing tube may have an inner diameter that is greater than an outer diameter of the inner sealing tube. The outer sealing tube may terminate at the same position as the inner sealing tube, as measured in the axial direction. Thus, the axial support structure can effectively support the outer wall to inhibit movement in the axial direction. Of course, in other embodiments a variation on the outer sealing tube could be used to achieve the same effect, for example a tube is not always necessary and could be replaced for example with a series of support members arranged around the circumference of the outer wall at positions inside the outer diameter of the outer wall.

The axial support structure may comprise an axial stop. The axial stop may be configured to abut the outer wall to inhibit movement of the inlet valve in the axial direction. The axial stop may be configured to abut the outer wall on a side opposite from the inner sealing tube and/or outer sealing tube. The axial stop may abut the outer wall at positions which are between the outer sealing tube and the inner sealing tube. The axial stop may comprise one or more stops, and preferably 4 or more stops for example eight stops. The one or more stops may be arranged an equal distance from the centre of the outer wall. Each stop may be arranged a distance from the centre of the outer wall that is between the outer sealing tube and inner sealing tube. The one or more stops may be spread equally in a circular shape. The one or more stops may be spread equally in a shape that matches the perimeter of the outer wall. Thus, the axial stop and stops may provide support to the inlet valve by ensuring the outer wall is held securely between by the axial support structure.

The wetting mechanism may comprise a base. The base may comprise the inner sealing tube. The base may comprise the outer sealing tube. The inner sealing tube may be configured to inhibit movement of the inlet valve away from the centre of the body in the axial direction. The outer sealing tube may be configured to inhibit movement of the inlet valve away from the centre of the body in the axial direction. The body may comprise the axial stop. The axial stop may be configured to inhibit movement of the inlet valve towards the centre of the body in the axial direction.

The wetting chamber may be tubular. The body may comprise an opening. The opening may be sized to allow the catheter to pass through the base. The opening may be sized to allow the catheter to pass into the wetting chamber. The sealing element may comprise a material that is more flexible than the body and/or the base. The sealing element may be resiliently deformed by the body and base, preferably to provide a fluid-tight seal between the body and the base.

According to another broad aspect of the invention there is provided a catheter assembly comprising: a catheter comprising a proximal end for insertion into the body and a distal end, and a wetting mechanism, wherein the wetting mechanism comprises a body, a base and a sealing element, the body defining a tubular wetting chamber through which the catheter may be moved to wet the catheter, the base comprising an opening sized to allow the catheter to pass through the base and into the wetting chamber, the sealing element comprising a material that is more flexible than the body and the base, and wherein the sealing element is resiliently deformed by the body and base to provide a fluid-tight seal between the body and the base.

The wetting mechanism may be arranged at the proximal end of the catheter. The sealing element may comprise an inlet valve configured to allow passage of the catheter into the wetting chamber. The inlet valve may be configured to inhibit release of fluid from the wetting chamber.

According to a third aspect of the invention there is provided a catheter assembly comprising: a catheter comprising a proximal end for insertion into the body and a distal end, and a wetting mechanism arranged at the proximal end of the catheter, wherein the wetting mechanism comprises a body, a base and a sealing element, the body defining a tubular wetting chamber through which the catheter may be moved to wet the catheter, the base comprising an opening sized to allow the catheter to pass through the base and into the wetting chamber, the sealing element comprising an inlet valve configured to allow passage of the catheter into the wetting chamber and inhibit release of fluid from the wetting chamber, the sealing element comprising a material that is more flexible than the body and the base, and wherein the sealing element is resiliently deformed by the body and base to provide a fluid-tight seal between the body and the base.

Advantageously, the body, base and sealing element provide a simple yet effective three-part wetting mechanism through the resilient deformation of the sealing element between the body and base. This allows the wetting mechanism to be easily constructed without sacrificing performance as the body and base can easily clip together to both structurally support the inlet valve and ensure a fluid-tight seal with the sealing element. This also allows the wetting mechanism to be constructed without welding parts of it together or use of adhesive, further reducing complexity and raw materials usage during manufacture. Furthermore, as the wetting mechanism is arranged at the proximal end of the catheter, this helps ensure the proximal end of the catheter is adequately wetted which is important given it is the first part of the catheter to enter the body in use.

The body, base and sealing element are thus preferably independent and not integrally formed.

The outer wall/outer support may be configured to provide a seal between the body and the sealing element. The outer wall/outer support may be configured to provide a seal between the base and the sealing element. The outer support may comprise an outer flange. The outer flange may extend in the axial direction. The outer flange may surround the entire outer wall in the transverse plane. The outer flange may be frustoconical. A narrow end of the outer flange may be attached to the outer wall, preferably the outer edge of the outer wall. A wide end of the outer flange may be at least 1%, 2%, 3%, or 5% larger in diameter than the narrow end. The wide end of the outer flange may be no more than 5%, 3%, 2%, or 1% larger in diameter than the narrow end. Preferably, the wide end of the outer flange has a diameter 2% larger than the narrow end. In one example, the narrow end of the outer flange has a diameter of about 13.5 mm and the wide end a diameter of about 14 mm. The outer flange may have a length between the narrow end and the wide end measured in the axial direction of at least 15%, 20%, 25% or 30% the outer diameter of the outer wall. The outer flange may have a length of no more than 30%, 25%, 20% or 15% the outer diameter of the outer wall. Preferably the outer flange has a length of 20% the outer diameter of the outer wall, or 60% longer than the length of the inner flange, for example about 3 mm. Thus, the outer flange provides a surface that extends in the axial direction as well as around the outer wall, similar to the inner flange. This can advantageously be abutted by the wetting mechanism to effectively inhibit movement of the outer wall and thereby the inlet valve in the transverse plane and axial direction without the inlet valve being obstructed by the wetting mechanism.

The base and/or body may be configured to receive the outer support/outer flange. The outer flange may be compressed by the wetting mechanism, preferably between the base and body. The body may comprise a slot configured to receive the outer flange. The slot may be the same shape as the outer support/outer flange, for example circular. The slot may be configured to resiliently deform the sealing element/outer flange. The slot may comprise an inner diameter that is between the diameters of the narrow end and wide end of the outer flange respectively. The slot may comprise an outer diameter which may be greater than the inner diameter by an amount equal to or preferably less than the thickness of the outer flange. An inner edge of the slot may be chamfered to assist the outer flange entering the slot. Thus, the slot can effectively seal between the body and sealing element as the outer flange is pressed inwardly as it is received in the slot as well as being compressed between the inner/outer diameter of the slot. This also helps to inhibit movement of the inlet valve.

At least part of the sealing element may deformed, for example compressed, in a radial direction by the base and body. The outer wall may be compressed in the radial direction by the wetting mechanism. The radial direction may be perpendicular to the axial direction, for example parallel to the transverse plane (and passing through the axis defining the axial direction). An inner diameter of the body may be less than an outer diameter of the outer flange. An inner diameter of the inner sealing tube may be less than an outer diameter of the inner flange. Thus, the inner flange and outer flange are urged inwards when abutted by the body and base such that the outer wall is compressed. This further assists with ensuring a fluid-tight seal and inhibiting unintentional movement of the inlet valve.

At least part of the sealing element may be compressed in the axial direction by the base and/or body. The outer support, and preferably the outer flange, may be compressed in the axial direction by the base and/or body. The slot may have a length in the axial direction. A distance between the closed end of the slot and the outer sealing tube may be less than the length of the outer flange. A distance between the closed end of the slot and the inner sealing tube may be less than the length of the outer flange. Thus, the outer sealing tube and/or inner sealing tube co-operate with the slot to urge the outer flange into the slot and compress it in the slot which helps ensure a fluid-tight seal while inhibiting unintentional movement of the inlet valve.

The outer support may comprise a sealing member. The sealing member may comprise a protrusion from the outer support, for example from the outer flange. The sealing member may provide a region of the outer support that is wider or thicker. The sealing member may have a thickness of at least 10%, 20%, 25%, 30% or 40% the thickness of the outer flange. The sealing member may have a thickness of no more than 40%, 30%, 25%, 20% or 10% the thickness of the outer flange. The outer flange in the region of the sealing member may have an outer diameter that is at least 5%, 10%, 15%, 20% or 25% wider than the diameter of the wide end of the outer flange.

The outer flange in the region of the sealing member may have an outer diameter that is no more than 25%, 20%, 15%, 10% or 5% wider than the diameter of the wide end of the outer flange. The outer flange may have an inner diameter that is made an equivalent amount smaller by the sealing member. The sealing member may be configured to be resiliently deformed by the wetting mechanism. The sealing member may be resiliently deformed by the body, for example within the slot. The sealing member may be a sealing rib. The sealing rib may extend around at least part of the perimeter of the outer flange, and preferably the entire perimeter. The sealing rib may be a semi-circular protrusion. Thus, the sealing member can be used to ensure resilient deformation of the outer flange within the slot to help provide a fluid-tight seal and additional support for the inlet valve.

The body may be tubular. The body may be cylindrical. The body may have open ends. The body may be configured to receive the outer sealing tube. The body may have an inner diameter that is substantially equal to an outer diameter of the outer sealing tube. The inner diameter of the body may be substantially equal to the outer diameter of the slot. Thus, the outer sealing tube may be received by the body and urge the sealing element into the slot.

The body may be configured to engage the base. The base may be configured to engage the body. The body and the base may not be separatable, for example during normal use of the wetting mechanism. Thus, the wetting mechanism may be robustly held together ensuring the catheter can be properly wetted and safely used.

The wetting mechanism may comprise two or more interlocking members. The interlocking members may be configured to secure the base and body together. The base may comprise at least one interlocking member. The body may comprise at least one interlocking member. At least one interlocking member may be a locking slot. At least one interlocking member may be a locking protrusion. The locking protrusion may be received in the locking slot. Each locking protrusion may correspond to a locking slot. Preferably, the wetting mechanism comprises at least two locking protrusions engageable with at least two locking slots. Thus, the interlocking members provide a simple and effective way for the body and base to be secured together in order to seal the wetting chamber.

The wetting mechanism may comprise two or more pairs of interlocking members. For example, four or more interlocking members, such as two or more locking protrusions engageable with two or more locking slots. Having at least two pairs of interlocking members helps ensure the device is more securely held and provides redundancy.

The two or more pairs of interlocking members may be spaced apart around the circumference of the wetting mechanism. One or more gaps may be provided between adjacent pairs of interlocking members to allow movement of at least one interlocking member. This ensures secure fitting around the wetting mechanism but also makes the wetting mechanism easier to fit together as the spacing and especially gaps allows the interlocking members to move more freely into engagement with one another.

At least one interlocking member may extend only part of the way around the circumference of the wetting mechanism. One, or preferably both, interlocking members in each pair may extend only part of the way around the circumference of the wetting mechanism. Thus, the interlocking members can move during device manufacture to make fitting the wetting mechanism together easier.

The at least one locking protrusion may be arranged on the base. The at least one locking protrusion may be arranged on the outer sealing tube. Each locking protrusion may span an arc length around the outer sealing tube equivalent to at least 50, 60, 70 or 80 degrees. Each locking protrusion may span an arc length around the outer sealing tube equivalent to no more than 80, 70, 60 or 50 degrees. The spacing between adjacent locking protrusions may be equal. There may be two locking protrusions on opposite sides of the wetting mechanism. Preferably, each locking protrusion spans an arc length of 65 degrees around the outer sealing tube. The at least one locking protrusion may be configured to urge the base into engagement with the base.

Each locking protrusion may be wedge-shaped. Each locking protrusion may have a wedge-shaped profile. Each locking protrusion may have a minimum thickness at an end distal from the base. Each locking protrusion may have a maximum thickness at an end proximal to the base. The thickness of each locking protrusion may vary linearly between the minimum and maximum thickness of the locking protrusion. Each locking protrusion may have a region of maximum thickness that spans at least 20%, 30% or 40% a length in a direction parallel to the axial direction of the locking protrusion. Each locking protrusion may have a region of maximum thickness that spans no more than 40%, 30% or 20% the length of the locking protrusion. The minimum thickness of each locking protrusion may be substantially zero, that is it does not increase the thickness of the object it is arranged on, such as the outer sealing tube. The maximum thickness of each locking protrusion may be at least 0.5 mm, 1 mm, 2 mm or 4 mm. The maximum thickness of each locking protrusion may be no more than 4 mm, 2 mm, 1 mm or 0.5 mm. The maximum thickness of each locking protrusion may be less than the thickness of the body. Each locking protrusion may extend only part of the way through a corresponding locking slot. Thus, the locking protrusions may be easily engaged into each slot and secure the body to the base without affecting the gripping protrusions of the cap outlined below.

Each locking protrusion may be mounted on a locking tongue. Each locking tongue may be configured to flex to allow the locking protrusion to move into engagement with a respective slot. The outer sealing tube may comprise one or more locking tongues corresponding to the one or more locking protrusions. Each locking tongue may be arranged at a free end of the outer sealing tube. Each locking tongue may be configured to abut the outer wall of the sealing element. Each locking tongue may be defined by a pair of gaps provided in the outer sealing tube. Each gap may extend from the free end of the outer sealing tube in the axial direction. Each gap may cover an arc length around the outer sealing tube equivalent to at least 5 degrees, 10 degrees or 15 degrees. Each gap may cover an arc length around the outer sealing tube equivalent to no more than 15 degrees, 10 degrees or 5 degrees. Each locking protrusion may span a majority of the arc length spanned by a locking tongue, and preferably the entire arc length. Each locking tongue may comprise at least one locking protrusion. Thus, the locking protrusions may easily move to be accommodated by a locking slot as they are mounted on a locking tongue. Each locking slot may span an arc length around the body equivalent to at least 50, 60, 70 or 80 degrees. Each locking slot may span an arc length around the body equivalent to no more than 80, 70, 60 or 50 degrees. The spacing between adjacent locking slots may be equal. There may be two locking slots on opposite sides of the wetting mechanism. Preferably, each locking slot spans an arc length of 65 degrees around the outer sealing tube. The at least one locking slot may be configured to urge the base into engagement with the base. Thus, the base and body may be easily and securely secured together by inserting the outer sealing tube into the body until the locking protrusions engage the locking slot.

Of course, while the locking slots are described as part of the body and the locking protrusions as part of the base, the relationship could also be reversed if required.

The base may comprise a skirt. The skirt may span between the inner sealing tube and outer sealing tube. The inner sealing element may extend from the skirt. The outer sealing element may extend from the skirt. The skirt may comprise an outer diameter that is greater than an outer diameter of the outer sealing tube. The skirt may comprise an outer diameter that is substantially equal to an outer diameter of the body. The skirt may be annular. The skirt may have an inner diameter that is no more than the inner diameter of the inner sealing tube. The outer diameter of the skirt may be at least 1.5 times, 2 times or 2.5 times larger than its inner diameter. The outer diameter of the skirt may be no more than 3 times, 2.5 times or 2 times larger than its inner diameter. Thus, the skirt provides structural support to the inner sealing tube and outer sealing tube as well as ensuring the base fits smoothly with the body.

The base may comprise an insertion tube. The insertion tube may provide the opening in the base. The insertion tube may be configured to guide passage of the catheter through the base and corresponding sealing element. The insertion tube may be arranged in the centre of the skirt. The insertion tube may extend from the skirt, preferably on a side opposite from the inner sealing tube and/or outer sealing tube. The insertion tube may be cylindrical. The insertion tube may have an inner diameter that is greater than the outer diameter of the catheter. The insertion tube may have an inner diameter that is at least as big as the outer diameter of the inlet valve. The insertion tube may have an outer diameter that corresponds to the inner diameter of the skirt. Thus, the insertion tube can be used to aid passage of the catheter through the base.

The skirt may be planar, for example substantially planar. The skirt may be curved. The skirt may be domed. The insertion tube may extend from a convex face of the skirt. Thus, the skirt may be used to prevent over insertion of the insertion tube into the body where it is used to deliver the catheter directly into the body.

The insertion tube may be open-ended. The insertion tube may be capped. The insertion tube may be capped with a dome, for example a hemicylindrical dome. The dome may comprise at least one slit, for example two slits. The two slits may be arranged in an orthogonal arrangement. The two slits may form a cross. The at least one slit may define at least two flaps, for example four flaps. The flaps may be configured to separate to allow the catheter to pass through the dome. Thus, the insertion tube may be capped to prevent dirt from entering the insertion tube and rendering the catheter unsafe for use. It also helps prevent any leakage of fluid from the wetting mechanism in the event it leaks through one of the valves. In addition, the domed shape and slits enable the insertion tube to be easily and comfortably used to direct the catheter into the body.

Each end of the body may be configured to receive a sealing element. Each end of the body may be configured to engage a body. Each end of the body may be substantially identical. The wetting mechanism may comprise two sealing elements. The two sealing elements may comprise a proximal sealing element and a distal sealing element. The distal sealing element may comprise the inlet valve. The proximal sealing element may comprise an outlet valve. The outlet valve may be configured to allow passage of the catheter out of the wetting chamber. The outlet valve may have any one or more of the features of the valve, the slit valve or the inlet valve as described above. Preferably, the outlet valve is identical or substantially identical to the inlet valve. Preferably, the outlet valve is a slit valve. The wetting mechanism may comprise two bases. Two sealing elements and two bases may seal both ends of the body. One of the bases may be an inserter tip. The inserter tip may comprise an insertion tube that is capped as described above. The inserter tip may be configured to engage one end of the body with the proximal sealing element sealed therebetween. The inserter tip may be configured to aid insertion of the catheter into the body, for example it may be shaped to aid insertion of the catheter into the body. The other base may be configured to engage the other end of the body with the distal sealing element sealed therebetween. Thus, the wetting mechanism can be easily constructed with parts that are similar and work together in a simple manner.

Each of the inlet valve and outlet valve may have a curved surface. Preferably each of the inlet valve and outlet valve are domed. The inlet and outlet valve may be arranged in the wetting mechanism with opposing curvature. The inlet and outlet valve may both be convex on a side facing away from the wetting chamber. Thus, the inlet and outlet valve are better arranged to retain liquid inside the wetting chamber and also provide additional space inside the wetting chamber for the wetting fluid/liquid.

The body may have a diameter of 10-20 mm, for example about 16 mm. The body may have a length of 20-30 mm, for example about 25 mm. The body may comprise at least one rib. Each rib may extend in the axial direction along the body. Each rib may have a length in the axial direction of at least 50%, 60%, 70% or 80% the length of the body. Each rib may have a length of no more than 80%, 70%, 60% or 50% the length of the body. Preferably, each rib has a length of 65% the body. Each rib may be joined to an inside of the body. Each rib may be joined to the body along a majority of its length, but preferably not all of its length. Each rib may extend inward from an inner diameter of the body by 15-25% of the inner diameter of the body. Preferably, the body may comprise at least two ribs, for example eight ribs. The ribs may be arranged at equal separations around the inside circumference of the body. Thus, the ribs can provide additional structural support to the body without significantly reducing the internal volume of the body.

Each rib may comprise a sealing slot provided between an end of the rib and the body. Preferably, each rib comprises a sealing slot at either end of the rib. Each sealing slot may be configured to receive the outer flange. The sealing slots at the same end of the at least one ribs may together form the slot mentioned above. Thus, the ribs can also urge the outer flange into engagement with the body to seal the wetting chamber.

The end of each rib may provide a stop as mentioned above. Thus, where the body comprises at least two ribs, the ribs together may provide the axial stop mentioned above. Thus the ribs can be used to efficiently inhibit movement of the outer wall and inlet valve.

The wetting mechanism may comprise a cap. The cap may be configured to overlie the base, for example the inserter tip. The cap may be provided over and around the inserter tip. The cap may comprise a shell that is slightly larger than the base/inserter tip so as to overlie it. Thus, the base/inserter tip is protected until use.

The cap may comprise a pull-ring. The pull-ring may be configured to allow a user to grasp the cap and pull it from the wetting mechanism. The pull-ring may of course be replaced by some other easily gripped feature that allows the cap to be easily removed from the wetting mechanism.

The cap may comprise a cap flange that extends around the body. The cap may comprise at least one interlocking member configured to engage the at least one interlocking member present on the body and/or base. The cap may comprise one or more gripping protrusions. The one or more gripping protrusions may be positioned on the cap flange. The one or more gripping protrusions may be configured to engage the one or more locking slots of the body. Each gripping protrusion may extend only part of the way through a corresponding locking slot. Thus, the cap is securely held on the wetting mechanism until it needs to be used without affecting the performance of the locking protrusions.

The cap, base and/or body may be formed from a material that is more rigid than the sealing element, for example high density polyethylene. The cap, base and/or body are preferably formed of a different material from the sealing element.

The catheter assembly may comprise a fluid collection bag arranged to receive liquid from the distal end of the catheter. The catheter assembly may therefore be a closed catheter assembly in that liquid released from the bladder is collected by the fluid collection bag. The fluid collection bag may comprise two panels joined about their periphery. The fluid collection bag may be any suitable shape or size. The fluid collection bag may be rectangular. The fluid collection bag may form a volume capable of storing 700-1000 ml of liquid.

The catheter may comprise a funnel arranged at the distal end of the catheter. The funnel may be attached to the fluid collection bag. The funnel may arranged within the fluid collection bag. A fluid-tight seal may be provided between the funnel and the fluid collection bag. The funnel may be configured to deliver liquid from the distal end of the catheter into the fluid collection bag. The sleeve may be attached to the funnel. The sleeve may be attached to the funnel by any suitable method such as: a weld; mechanical seal; heat seal; pressure seal; adhesive; solvent bond; ultraviolet bond; ultrasonic weld; laser weld; impulse weld; or friction weld. A fluid-tight seal may be provided between the sleeve and the funnel. The funnel may comprise bypass tubes to provide a fluid connection between the sleeve and fluid collection bag. Thus, liquid may efficiently pass from the catheter and/or sleeve into the fluid collection bag without leaking outside the catheter assembly.

Alternatively, the catheter assembly may be configured to allow fluid to flow out of the catheter assembly. In such embodiments, the catheter assembly is an “open catheter assembly”. The funnel may be configured to direct the flow of fluid out of the catheter assembly (for example into a lavatory or the like). The catheter assembly may, therefore, not comprise a fluid collection bag.

The sleeve may comprise a flexible plastics material. The sleeve may be liquid impermeable. The sleeve may comprise a thermoplastic polyurethane (TPU) or low-density polyethylene (LDPE). The sleeve may be attached to the base, for example the outside of the insertion tube of the base. The sleeve may be attached via any suitable method such as: a weld; mechanical seal; heat seal; pressure seal; adhesive; solvent bond; ultraviolet bond; ultrasonic weld; laser weld; impulse weld; or friction weld. Thus, the sleeve is cheap and easy to produce and can be easily manipulated by the user during use.

The catheter may be formed of a hydrophilic material. The catheter surface may be activatable by a water-based fluid, for example water. The wetting chamber may be configured to contain a wetting liquid, for example water, or a polar/water-based wetting liquid. The wetting liquid may have a viscosity of no more than 10,000 centipoise, or no more than 1,000 centipoise, or no more than 100 centipoise, or no more than 10 centipoise. The slit valve may therefore be configured to inhibit the release of the wetting liquid from the wetting chamber. This ensures the wetting liquid can efficiently wet the catheter.

The catheter may be formed of a material of the group comprising: polyvinyl chloride, polytetrafluoroethylene, polyolefins, latex, silicones, synthetic rubbers, polyurethanes, polyesters, polyacrylates, polyamides, thermoplastic elastomeric materials, styrene block copolymers, polyether block amide, thermoplastic vulcanizates, thermoplastic copolyesters, thermoplastic polyamides, and water disintegrable or enzymatically hydrolysable material, or combinations, blends or co-polymers of any of the above materials.

The water disintegrable or enzymatically hydrolysable material may comprise a material of the group comprising: polyvinyl alcohol, extrudable polyvinyl alcohol, polyacrylic acids, polylactic acid, polyesters, polyglycolide, polyglycolic acid, poly lactic-co-glycolic acid, polylactide, amines, polyacrylamides, poly(N-(2-Hydroxypropyl)methacrylamide), starch, modified starches or derivatives, amylopectin, pectin, xanthan, scleroglucan, dextrin, chitosans, chitins, agar, alginate, carrageenans, laminarin, saccharides, polysaccharides, sucrose, polyethylene oxide, polypropylene oxide, acrylics, polyacrylic acid blends, poly(methacrylic acid), polystyrene sulfonate, polyethylene sulfonate, lignin sulfonate, polymethacrylamides, copolymers of aminoalkyl-acrylamides and methacrylamides, melamine-formaldehyde copolymers, vinyl alcohol copolymers, cellulose ethers, poly-ethers, polyethylene oxide, blends of polyethylene-polypropylene glycol, carboxymethyl cellulose, guar gum, locust bean gum, hydroxypropyl cellulose, vinylpyrrolidone polymers and copolymers, polyvinyl pyrrolidone-ethylene-vinyl acetate, polyvinyl pyrrolidone-carboxymethyl cellulose, carboxymethyl cellulose shellac, copolymers of vinylpyrrolidone with vinyl acetate, hydroxyethyl cellulose, gelatin, poly-caprolactone, poly(p-dioxanone), or combinations, blends or co-polymers of any of the above materials.

Preferably, the catheter is formed of a polyolefin material, in particular polyethylene and/or polypropylene.

Preferably, the catheter is formed of a thermoplastic elastomeric material.

The catheter may be a urinary catheter. The catheter may be a male urinary catheter. The catheter may be a female urinary catheter. The catheter may be an intermittent catheter. In one embodiment, the catheter is an intermittent male urinary catheter. Thus, the features of the present invention allow intermittent male urinary catheters to be adequately wetted prior to use which can be more difficult than for other types of catheter which are generally shorter.

The catheter assemblies of the first to third aspects may include any one or more features of a catheter assembly as defined in general/broad terms, or according to any other of the first to third aspects set out above. The catheter assemblies of the first to third aspects may comprise any of the optional features of the others of the first to third aspects without necessarily including all the features required of them. That is to say, an optional feature which happens to be set out following one particular aspect does not necessarily apply only to that aspect, so, for example, the disclosure provides for a catheter assembly comprising: a catheter assembly comprising: a catheter comprising a proximal end for insertion into the user and a distal end, and a wetting mechanism arranged at the proximal end of the catheter, wherein the wetting mechanism comprises a body, the body defines a wetting chamber through which the catheter may be moved to wet the catheter, and wetting mechanism comprises a slit valve configured to allow passage of the catheter into the wetting chamber and inhibit release of fluid from the wetting chamber as described in relation to the first aspect and wherein the wetting mechanism comprises a base comprising a curved skirt as described in relation to the third aspect.

In a preferred embodiment, there is thus provided a catheter assembly comprising: a catheter comprising a proximal end for insertion into the body and a distal end, and a wetting mechanism arranged at the proximal end of the catheter, wherein the wetting mechanism comprises a body, the body defines a wetting chamber through which the catheter may be moved to wet the catheter, and the wetting mechanism comprises a slit valve configured to allow passage of the catheter into the wetting chamber and to inhibit release of fluid from the wetting chamber, wherein the catheter is formed of a hydrophilic material and the wetting chamber is configured to contain a wetting liquid, wherein the slit valve is configured to inhibit the release of the wetting liquid from the wetting chamber.

In a preferred embodiment, there is thus provided a catheter assembly comprising: a catheter comprising a proximal end for insertion into the body and a distal end, and a wetting mechanism arranged at the proximal end of the catheter, wherein the wetting mechanism comprises a body, the body defines a wetting chamber through which the catheter may be moved to wet the catheter, wherein the wetting mechanism comprises two slit valves: an inlet valve and an outlet valve, the inlet valve configured to allow passage of the catheter into the wetting chamber and to inhibit release of fluid from the wetting chamber, and the outlet valve configured to allow passage of the catheter out of the wetting chamber and inhibit release of fluid from the wetting chamber, further comprising an inserter tip configured to engage one end of the body with the outlet valve therebetween and shaped to aid insertion of the catheter into the body.

In a preferred embodiment, there is thus provided a catheter assembly comprising: a catheter comprising a proximal end for insertion into the body and a distal end, and a wetting mechanism arranged at the proximal end of the catheter, wherein the wetting mechanism comprises a body, the body defines a wetting chamber through which the catheter may be moved to wet the catheter, wherein the wetting mechanism comprises two slit valves: an inlet valve and an outlet valve, the inlet valve configured to allow passage of the catheter into the wetting chamber and to inhibit release of fluid from the wetting chamber, and the outlet valve configured to allow passage of the catheter out of the wetting chamber and inhibit release of fluid from the wetting chamber, wherein each of the inlet and outlet valve have a curved surface and the inlet and outlet valve are arranged in the wetting mechanism with opposing curvature.

In a preferred embodiment, there is thus provided a catheter assembly comprising: a catheter comprising a proximal end for insertion into the body and a distal end, and a wetting mechanism arranged at the proximal end of the catheter, wherein the wetting mechanism comprises a body, the body defines a wetting chamber through which the catheter may be moved to wet the catheter, and the wetting mechanism comprises a slit valve configured to allow passage of the catheter into the wetting chamber and to inhibit release of fluid from the wetting chamber, wherein the slit valve has a curved surface and is preferably domed.

In a preferred embodiment, there is thus provided a catheter assembly comprising: a catheter comprising a proximal end for insertion into the body and a distal end, and a wetting mechanism arranged at the proximal end of the catheter, wherein the wetting mechanism comprises a body, the body defines a wetting chamber through which the catheter may be moved to wet the catheter, and the wetting mechanism comprises a slit valve configured to allow passage of the catheter into the wetting chamber and to inhibit release of fluid from the wetting chamber, wherein the slit valve has a curved surface and is convex on a side facing away from the wetting chamber.

In a preferred embodiment, there is thus provided a catheter assembly comprising: a catheter comprising a proximal end for insertion into the body and a distal end, and a wetting mechanism arranged at the proximal end of the catheter, wherein the wetting mechanism comprises a body, the body defines a wetting chamber through which the catheter may be moved to wet the catheter, and the wetting mechanism comprises a slit valve configured to allow passage of the catheter into the wetting chamber and to inhibit release of fluid from the wetting chamber, wherein the slit valve is domed and has a dome height of no more than 30% the diameter of the slit valve.

In a preferred embodiment, there is thus provided a catheter assembly comprising: a catheter comprising a proximal end for insertion into the body and a distal end, and a wetting mechanism arranged at the proximal end of the catheter, wherein the wetting mechanism comprises a body, a base and a sealing element, the body defining a tubular wetting chamber through which the catheter may be moved to wet the catheter, the base comprising an opening sized to allow the catheter to pass through the base and into the wetting chamber, the sealing element comprising an inlet valve configured to allow passage of the catheter into the wetting chamber and inhibit release of fluid from the wetting chamber, the sealing element comprising a material that is more flexible than the body and the base, and wherein the sealing element is resiliently deformed by the body and base to provide a fluid-tight seal between the body and the base, wherein the catheter comprises a hydrophilic material and the wetting chamber is configured to contain a wetting liquid.

In a preferred embodiment, there is thus provided a catheter assembly comprising: a catheter comprising a proximal end for insertion into the body and a distal end, and a wetting mechanism arranged at the proximal end of the catheter, wherein the wetting mechanism comprises a body, a base and a sealing element, the body defining a tubular wetting chamber through which the catheter may be moved to wet the catheter, the base comprising an opening sized to allow the catheter to pass through the base and into the wetting chamber, the sealing element comprising an inlet valve configured to allow passage of the catheter into the wetting chamber and inhibit release of fluid from the wetting chamber, the sealing element comprising a material that is more flexible than the body and the base, and wherein the sealing element is resiliently deformed by the body and base to provide a fluid-tight seal between the body and the base, wherein the sealing element comprises an outer flange extending in the axial direction and being compressed by the base and body.

In a preferred embodiment, there is thus provided a catheter assembly comprising: a catheter comprising a proximal end for insertion into the body and a distal end, and a wetting mechanism arranged at the proximal end of the catheter, wherein the wetting mechanism comprises a body, a base and a sealing element, the body defining a tubular wetting chamber through which the catheter may be moved to wet the catheter, the base comprising an opening sized to allow the catheter to pass through the base and into the wetting chamber, the sealing element comprising an inlet valve configured to allow passage of the catheter into the wetting chamber and inhibit release of fluid from the wetting chamber, the sealing element comprising a material that is more flexible than the body and the base, and wherein the sealing element is resiliently deformed by the body and base to provide a fluid-tight seal between the body and the base, wherein the sealing element has a top-hat profile.

In a preferred embodiment, there is thus provided a catheter assembly comprising: a catheter comprising a proximal end for insertion into the body and a distal end, and a wetting mechanism arranged at the proximal end of the catheter, wherein the wetting mechanism comprises a body, a base and a sealing element, the body defining a tubular wetting chamber through which the catheter may be moved to wet the catheter, the base comprising an opening sized to allow the catheter to pass through the base and into the wetting chamber, the sealing element comprising an inlet valve configured to allow passage of the catheter into the wetting chamber and inhibit release of fluid from the wetting chamber, the sealing element comprising a material that is more flexible than the body and the base, and wherein the sealing element is resiliently deformed by the body and base to provide a fluid-tight seal between the body and the base, wherein an axial direction is defined as the direction in which the catheter passes into the wetting chamber through the inlet valve, the sealing element comprises an inner flange and an outer flange both extending in the axial direction and separated by an outer wall, the body comprises a slot configured to receive the outer flange and the base comprises an inner sealing tube configured to abut the inner flange, wherein the distance measured in the radial direction between an inner diameter of the slot and an inner diameter of the inner sealing tube is greater than the distance between an outer diameter of the inner flange and an inner diameter of the outer flange.

In a preferred embodiment, there is thus provided a catheter assembly comprising: a urinary catheter comprising a proximal end for insertion into the body and a distal end, and a wetting mechanism arranged at the proximal end of the catheter; wherein the wetting mechanism comprises a wetting chamber and a sealing element, the sealing element comprising an inlet valve configured to allow passage of the catheter into the wetting chamber in an axial direction to be wetted by wetting fluid, the inlet valve is configured to inhibit release of fluid from the wetting chamber, the inlet valve defines a transverse plane perpendicular to the axial direction, the sealing element comprises: a stepped profile, an outer wall extending parallel to the transverse plane but offset from the inlet valve, an inner support extending in the axial direction and connecting the outer wall to the inlet valve, wherein the wetting mechanism abuts the inner support to inhibit movement of the inlet valve in the transverse plane, and the wetting mechanism abuts the outer wall to inhibit movement of the inlet valve in the axial direction.

In a preferred embodiment, there is thus provided a catheter assembly comprising: an intermittent male urinary catheter comprising a proximal end for insertion into the body and a distal end, and a wetting mechanism arranged at the proximal end of the catheter; wherein the wetting mechanism comprises a wetting chamber and a sealing element, the sealing element comprising an inlet valve configured to allow passage of the catheter into the wetting chamber in an axial direction to be wetted by wetting fluid, the inlet valve is configured to inhibit release of fluid from the wetting chamber, the inlet valve defines a transverse plane perpendicular to the axial direction, the sealing element comprises: a stepped profile, an outer wall extending parallel to the transverse plane but offset from the inlet valve, an inner support extending in the axial direction and connecting the outer wall to the inlet valve, wherein the wetting mechanism abuts the inner support to inhibit movement of the inlet valve in the transverse plane, and the wetting mechanism abuts the outer wall to inhibit movement of the inlet valve in the axial direction.

In a preferred embodiment, there is thus provided a catheter assembly comprising: a catheter comprising a proximal end for insertion into the body and a distal end, and a wetting mechanism arranged at the proximal end of the catheter; wherein the wetting mechanism comprises a wetting chamber and a sealing element, the sealing element comprising an inlet valve configured to allow passage of the catheter into the wetting chamber in an axial direction to be wetted by wetting fluid, the inlet valve is configured to inhibit release of fluid from the wetting chamber, the inlet valve defines a transverse plane perpendicular to the axial direction, the sealing element comprises: a stepped profile, an outer wall extending parallel to the transverse plane but offset from the inlet valve, an inner support extending in the axial direction and connecting the outer wall to the inlet valve, wherein the wetting mechanism abuts the inner support to inhibit movement of the inlet valve in the transverse plane, and the wetting mechanism abuts the outer wall to inhibit movement of the inlet valve in the axial direction, wherein the inner support comprises an inner flange surrounding the inlet valve in the transverse plane and the inner flange is frustoconical.

In a preferred embodiment, there is thus provided a catheter assembly comprising: a catheter comprising a proximal end for insertion into the body and a distal end, and a wetting mechanism arranged at the proximal end of the catheter; wherein the wetting mechanism comprises a wetting chamber and a sealing element, the sealing element comprising an inlet valve configured to allow passage of the catheter into the wetting chamber in an axial direction to be wetted by wetting fluid, the inlet valve is configured to inhibit release of fluid from the wetting chamber, the inlet valve defines a transverse plane perpendicular to the axial direction, the sealing element comprises: a stepped profile, an outer wall extending parallel to the transverse plane but offset from the inlet valve, an inner support extending in the axial direction and connecting the outer wall to the inlet valve, wherein the wetting mechanism abuts the inner support to inhibit movement of the inlet valve in the transverse plane, and the wetting mechanism abuts the outer wall to inhibit movement of the inlet valve in the axial direction, wherein the outer wall is annular and outer wall is compressed in a direction parallel to the transverse plane by the wetting mechanism.

In a preferred embodiment, there is thus provided a catheter assembly comprising: a catheter comprising a proximal end for insertion into the body and a distal end, and a wetting mechanism arranged at the proximal end of the catheter; wherein the wetting mechanism comprises a wetting chamber and a sealing element, the sealing element comprising an inlet valve configured to allow passage of the catheter into the wetting chamber in an axial direction to be wetted by wetting fluid, the inlet valve is configured to inhibit release of fluid from the wetting chamber, the inlet valve defines a transverse plane perpendicular to the axial direction, the sealing element comprises: a stepped profile, an outer wall extending parallel to the transverse plane but offset from the inlet valve, an inner support extending in the axial direction and connecting the outer wall to the inlet valve, wherein the wetting mechanism abuts the inner support to inhibit movement of the inlet valve in the transverse plane, and the wetting mechanism abuts the outer wall to inhibit movement of the inlet valve in the axial direction, wherein the outer wall further comprises an outer flange, the outer flange being compressed by the wetting mechanism.

According to a fourth aspect of the invention there is provided a method of manufacturing a catheter assembly comprising providing a catheter comprising a proximal end for insertion into the user and a distal end and a wetting mechanism, wherein the wetting mechanism comprises a body, the body defines a wetting chamber through which the catheter may be moved to wet the catheter, and wetting mechanism comprises a slit valve configured to allow passage of the catheter into the wetting chamber and inhibit release of fluid from the wetting chamber, the method comprising arranging the wetting mechanism at the proximal end of the catheter.

The method of the fourth aspect of the invention may be a method of manufacturing the catheter assembly of the first aspect of the invention, which, of course, may include any optional feature outlined above.

According to fifth aspect of the present invention there is provided a method of manufacturing a catheter assembly comprising providing a catheter comprising a proximal end for insertion into the body and a distal end and a wetting mechanism, wherein the wetting mechanism comprises a wetting chamber and a sealing element, the sealing element comprising an inlet valve configured to allow passage of the catheter into the wetting chamber in an axial direction to be wetted by wetting fluid, the inlet valve is configured to inhibit release of fluid from the wetting chamber, the inlet valve defines a transverse plane perpendicular to the axial direction, the sealing element comprising a stepped profile, the sealing element comprising an outer wall extending parallel to the transverse plane but offset from the inlet valve, the sealing element comprising an inner support extending in the axial direction and connecting the outer wall to the inlet valve, the wetting mechanism abuts the inner support to inhibit movement of the inlet valve in the transverse plane, and the wetting mechanism abuts the outer wall to inhibit movement of the inlet valve in the axial direction, the method comprising arranging the wetting mechanism at the proximal end of the catheter.

The method of the fifth aspect of the invention may be a method of manufacturing the catheter assembly of the second aspect of the invention, which, of course, may include any optional feature outlined above.

According to a sixth aspect of the invention there is provided a method of manufacturing a catheter assembly comprising providing a catheter comprising a proximal end for insertion into the body and a distal end and a wetting mechanism wherein the wetting mechanism comprises a body, a base and a sealing element, the body defining a tubular wetting chamber through which the catheter may be moved to wet the catheter, the base comprising an opening sized to allow the catheter to pass through the base and into the wetting chamber, the sealing element comprising a material that is more flexible than the body and the base, and wherein the sealing element is resiliently deformed by the body and base to provide a fluid-tight seal between the body and the base, the method comprising arranging the wetting mechanism at the proximal end of the catheter. The sealing element may comprise an inlet valve configured to allow passage of the catheter into the wetting chamber and inhibit release of fluid from the wetting chamber.

The method of the sixth aspect of the invention may be a method of manufacturing the catheter assembly of the third aspect of the invention, which, of course, may include any optional feature outlined above.

The method may comprise arranging the base, sealing element and body co-axially. The method may comprise fitting an inlet valve of the sealing element into the base. The method may comprise moving the base into engagement with the body with the sealing element therebetween. The method may comprise providing a wetting fluid into the body. The method may comprise providing a second base and second sealing element. The method may comprise fitting the second sealing element onto the body. The method may comprise sealing both ends of the body with two sealing elements and two bases. The method may comprise fitting the second base onto the body to seal the body and create the wetting chamber.

According to a seventh aspect of the invention there is provided a method of wetting a catheter, the catheter comprising a proximal end for insertion into the body and a distal end, the method comprising providing a wetting mechanism at the proximal end of the catheter, wherein the wetting mechanism comprises a slit valve and a body defining a wetting chamber comprising wetting fluid, the slit valve configured to inhibit release of wetting fluid from the wetting chamber, the method comprising moving the catheter through the slit valve and into the wetting chamber to wet the catheter.

The method of the seventh aspect of the invention may be a method of wetting a catheter from the catheter assembly of the first aspect of the invention, which, of course, may include any optional feature outlined above and may be manufactured according to the fourth aspect of the invention.

The method may include separating the flaps of the slit valve with the catheter.

The method may comprise inserting the insertion tube into the urethra. The method may comprise progressively moving the proximal end of the catheter through the wetting mechanism. The method may comprise introducing the catheter into the body, preferably via the insertion tube. The method may comprise allowing fluid to pass from the body and into the fluid collection bag via the catheter. Consequently, the method may be a method of using a catheter assembly.

According to an eighth aspect of the present invention there is provided a method of wetting a catheter comprising a proximal end for insertion into the body, the method comprising arranging a wetting mechanism at the proximal end of the catheter, wherein the wetting mechanism comprises a wetting chamber and a sealing element, the method comprising moving the catheter in an axial direction through an inlet valve in the sealing element and into the wetting chamber, wherein, the inlet valve is configured to inhibit release of fluid from the wetting chamber, the inlet valve defines a transverse plane perpendicular to the axial direction, the sealing element comprising a stepped profile, the sealing element comprises an outer wall extending parallel to the transverse plane but offset from the inlet valve, and an inner support extending in the axial direction and connecting the outer wall to the inlet valve, wherein the wetting mechanism abuts the inner support to inhibit movement of the inlet valve in the transverse plane, and the wetting mechanism abuts the outer wall to inhibit movement of the inlet valve in the axial direction.

The method of the eighth aspect of the invention may be a method of wetting a catheter in the catheter assembly of the second aspect of the invention, which, of course, may include any optional feature outlined above and may be manufactured according to the fifth aspect of the invention.

The method may comprise inserting the insertion tube into the urethra. The method may comprise progressively moving the proximal end of the catheter through the wetting mechanism. The method may comprise introducing the catheter into the body, preferably via the insertion tube. The method may comprise allowing fluid to pass from the body and into the fluid collection bag via the catheter. Consequently, the method may be a method of using a catheter assembly.

According to a ninth aspect of the present invention there is provided a method of wetting a catheter comprising a proximal end for insertion into the body and a distal end, the method comprising providing a wetting mechanism at the proximal end of the catheter, wherein the wetting mechanism comprises a body, a base and a sealing element, the method comprising moving the catheter through an opening in the base, through an inlet valve in the sealing element and into a tubular wetting chamber defined by the body, wherein the inlet valve is configured to inhibit release of fluid from the wetting chamber, the sealing element comprises a material that is more flexible than the body and the base, and wherein the sealing element is resiliently deformed by the body and base to provide a fluid-tight seal between the body and the base.

The method of the ninth aspect of the invention may be a method of wetting a catheter in the catheter assembly of the third aspect of the invention, which, of course, may include any optional feature outlined above and may be manufactured according to the sixth aspect of the invention.

The method may comprise inserting the insertion tube into the urethra. The method may comprise progressively moving the proximal end of the catheter through the wetting mechanism. The method may comprise introducing the catheter into the body, preferably via the insertion tube. The method may comprise allowing fluid to pass from the body and into the fluid collection bag via the catheter. Consequently, the method may be a method of using a catheter assembly.

The methods of the fourth to ninth aspects of the present invention may of course individually include any one or more of the features, optional or otherwise, of one another and further may include any one or more of the features, optional or otherwise, of the first to third aspects of the present invention.

### DETAILED DESCRIPTION OF THE INVENTION

In order that the invention may be more clearly understood one or more embodiments thereof will now be described, by way of example only, with reference to the accompanying drawings, of which:

FIG. 1 is a schematic side view of a first embodiment of a catheter assembly;

FIG. 2 is a side view of a distal sealing element of the catheter assembly of FIG. 1;

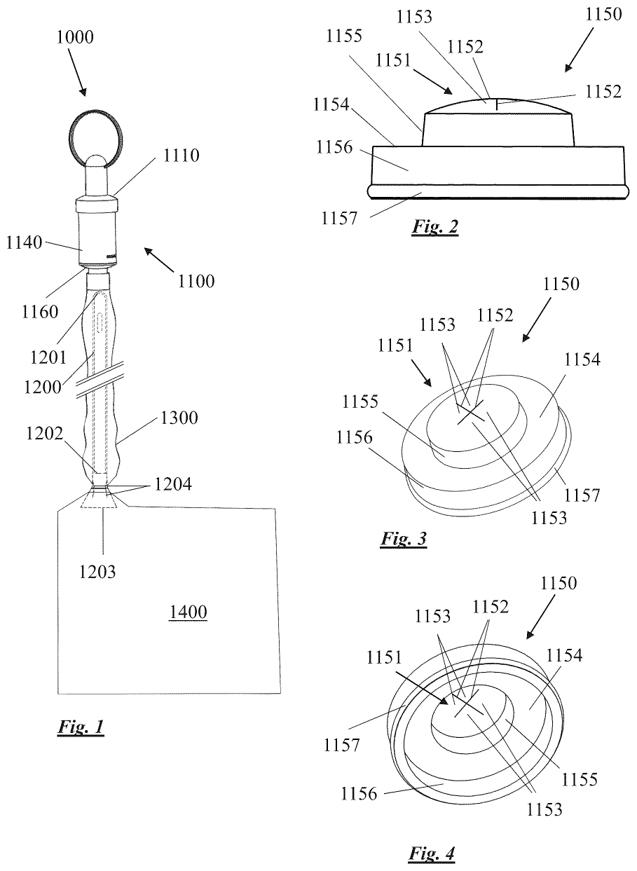

FIG. 3 is a top perspective view of the distal sealing element of FIG. 2;

FIG. 4 is a bottom perspective view of the distal sealing element of FIG. 2;

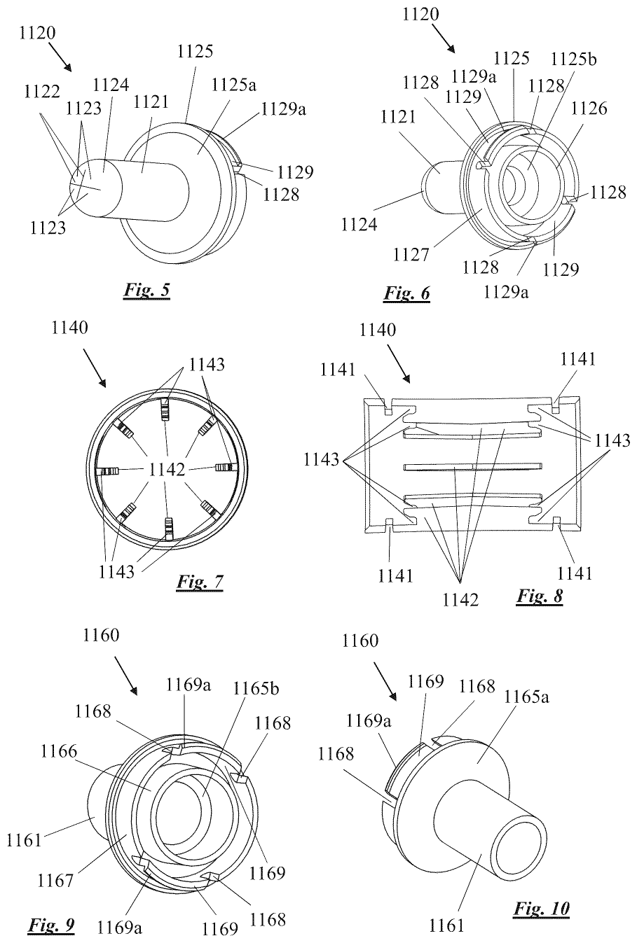

FIG. 5 is a top perspective view of a inserter tip of the catheter assembly of FIG. 1;

FIG. 6 is a bottom perspective view of the inserter tip of FIG. 5;

FIG. 7 is a top view of a body of the catheter assembly of FIG. 1;

FIG. 8 is a side cross-sectional view of the body of FIG. 7;

FIG. 9 is a bottom perspective view of a base of the catheter assembly of FIG. 1;

FIG. 10 is a top perspective view of the base of FIG. 9;

FIG. 11 is a side perspective view of a cap of the catheter assembly of FIG. 1;

FIG. 12 is a bottom perspective view of the cap of FIG. 11;

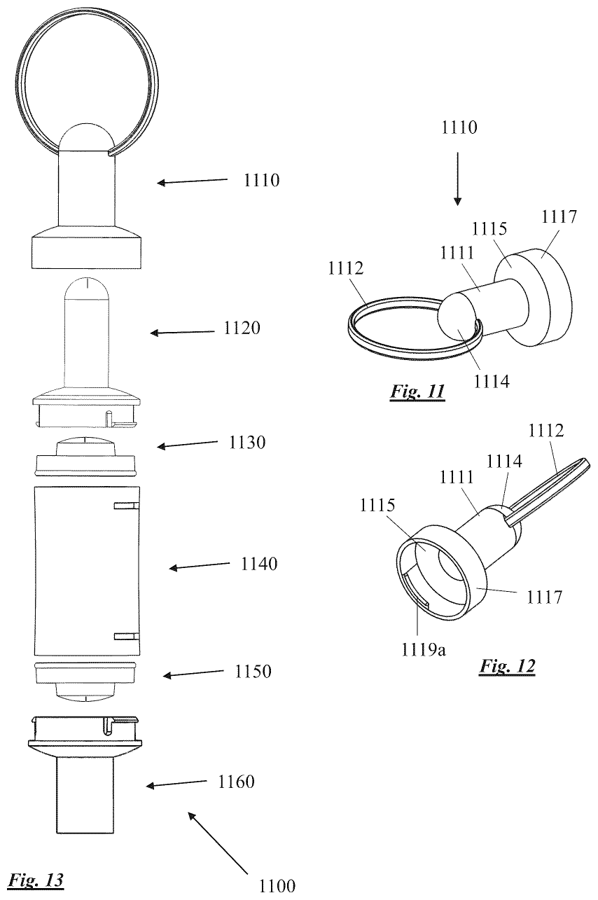

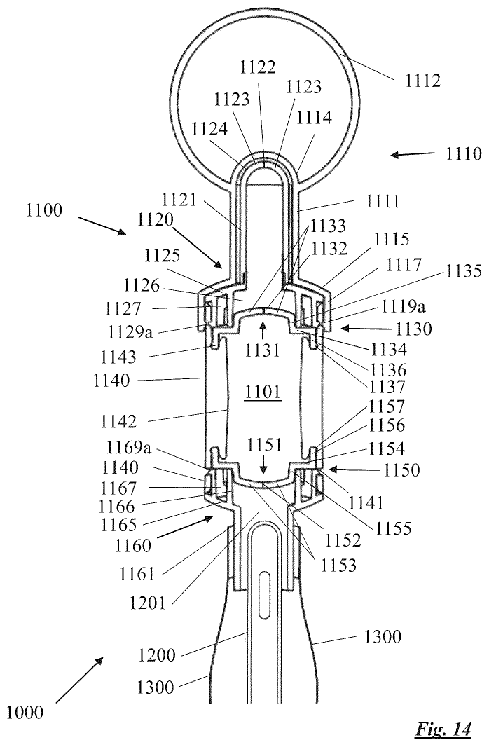

FIG. 13 is an exploded view of a wetting mechanism of the catheter assembly of FIG. 1;

FIG. 14 is a cross-sectional view of the catheter assembly of FIG. 1 before wetting of the catheter;

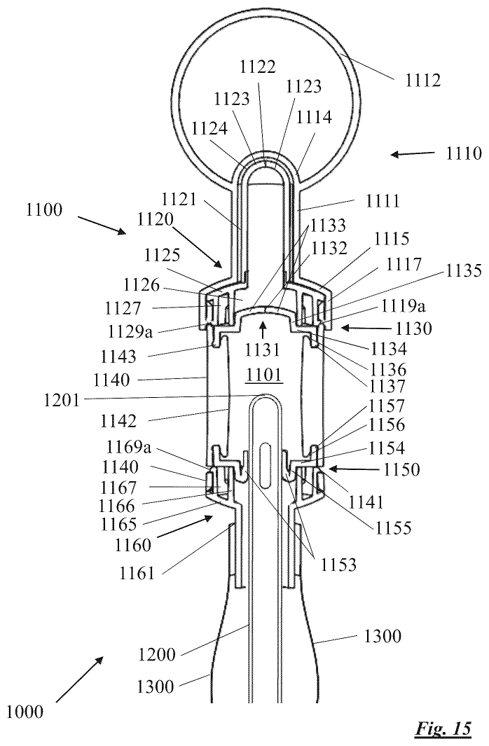

FIG. 15 is a cross-sectional view of the catheter assembly of FIG. 1 during wetting of the catheter;

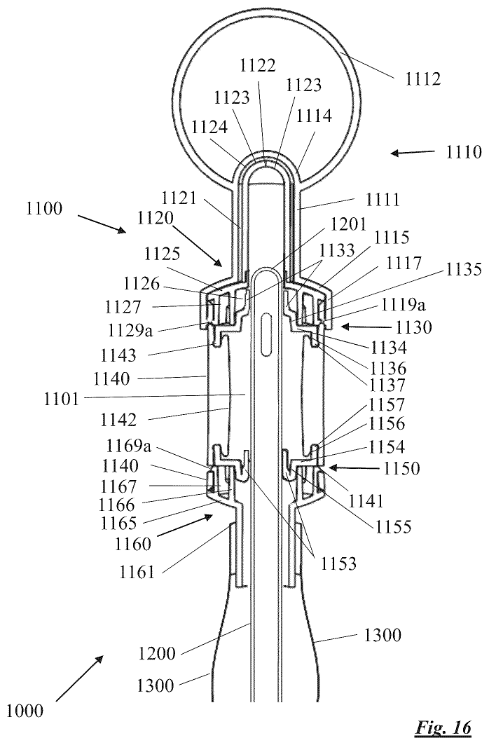

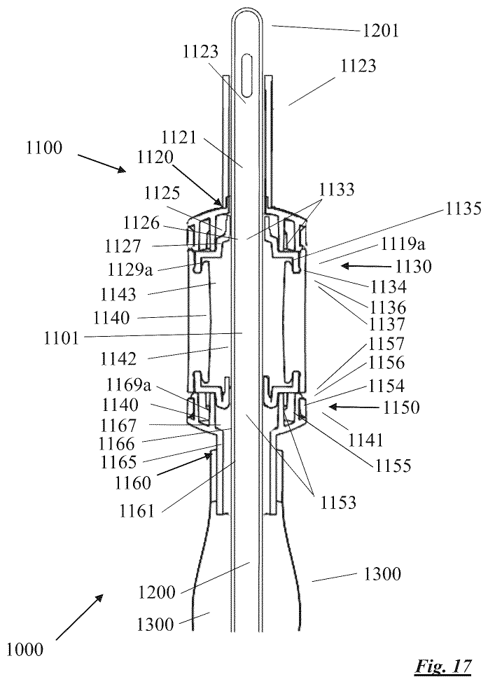

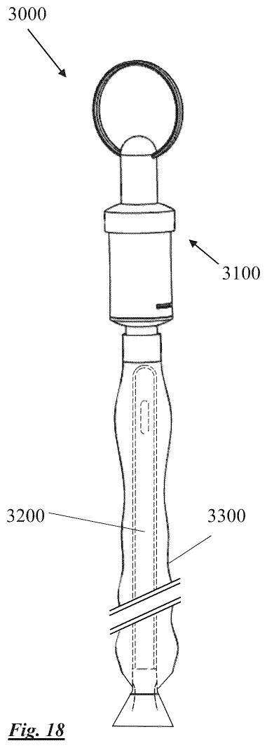

FIG. 16 is a cross-sectional view of the catheter assembly of FIG. 1 during wetting of the catheter;

FIG. 17 is a cross-sectional view of the catheter assembly of FIG. 1 during wetting of the catheter when the cap has been removed; and

FIG. 18 is a schematic side view of a second embodiment of a catheter assembly.

Referring to FIGS. 1-17, a first embodiment of a catheter assembly 1000 comprises a wetting mechanism 1100, a catheter 1200, a sleeve 1300 and a fluid collection bag 1400. The catheter 1200 comprises a proximal end 1201 for insertion into the user and a distal end 1202. In this embodiment, the catheter 1201 is a male urinary catheter made from a hydrophilic thermoplastic elastomer (TPE). The sleeve 1301 of this embodiment is a thermoplastic polyurethane (TPU) or low-density polyethylene (LDPE). Obviously those skilled in the art will be able to select suitable alternative materials.

In this embodiment, the wetting mechanism 1100 comprises a cap 1110, an inserter tip 1120, a proximal sealing element 1130, a body 1140, a distal sealing element 1150 and a base 1160.

The proximal and distal sealing elements 1130, 1150 are formed of a flexible material such as a flexible plastics material, rubber or silicone-in this embodiment, rubber is used. The cap 1110, inserter tip 1120, body 1140, and base 1160 comprise a material that is more rigid than the proximal and distal sealing elements 1130, 1150, for example a hard plastics material such as high density polyethylene (HDPE).

In this embodiment, the distal sealing element 1150 is generally sheet-like with a constant thickness unless mentioned otherwise below. The distal sealing element 1150 comprises an inlet valve 1151 arranged at the centre of the distal sealing element 1151. In this embodiment, the inlet valve 1151 is circular and domed with two slits 1152 at the centre. The two slits 1152 are orthogonally arranged and define a cross in the centre of the inlet valve 1151 defining four moveable flaps 1153. The inlet valve 1151 is therefore a normally closed slit valve openable through movement of the flaps 1153 away from one another. When the valve is closed, the flaps 1153 are in a closed position in which they co-operate to seal the inlet valve 1151. Of course, in other embodiments only one slit may be present (and two flaps) or two or more slits may be arranged in a different pattern from that described above with a corresponding number of flaps, for example a T-shaped slit arrangement may be used defining three flaps.

In this embodiment, each slit 1152 spans approximately 70% the diameter of the inlet valve 1151, such as 5-6 mm, for example 5.5 mm. In other embodiments, different lengths of slit may be used as appropriate. In this embodiment, the inlet valve 1151 has a diameter of 7-9 mm, for example about 8 mm, and is domed with a dome height that is approximately 10% of the diameter of the inlet valve 1151, that is about 1 mm in this embodiment.

In this embodiment, an axial direction is defined as the direction in which the catheter 1200 passes into the wetting chamber, for example the axial direction is parallel to the dome height of the inlet valve 1151. The inlet valve 1151 also defines a transverse plane perpendicular to the axial direction.

In this embodiment, the distal sealing element 1150 further comprises an annular outer wall 1154. The outer wall 1154 has an inner diameter that is slightly larger than the outer diameter of the inlet valve 1151, for example by approximately 5%, the inner diameter of the outer wall 1154 may therefore be about 8.5 mm. The outer wall 1154 has an outer diameter that is 73% larger than the outer diameter of the inlet valve 1151, for example about 14 mm. The outer wall 1154 is arranged co-axially with the circular inlet valve 1151 but is offset in the axial direction from the inlet valve 1151 by a distance approximately equal to 20-25% of the diameter of the inlet valve, for example 22% or about 2 mm. The outer wall 1154 and inlet valve 1151 are arranged such that the domed surface of the inlet valve 1151 curves down towards the outer wall 1151 at the edge of the inlet valve 1151.

In this embodiment, the distal sealing element 1150 comprises an inner support in the form of an inner flange 1155 that connects the outer edge of the inlet valve 1151 to the inner edge of the outer wall 1154 around the circumference of the inlet valve 1151. Due to the inner diameter of the outer wall 1154 being slightly larger than the outer diameter of the inlet valve 1151, the inner flange 1155 is an open-ended frustoconical shape extending in the axial direction. Of course, in other embodiments, the inner flange 1155 may not be frustoconical and could be any suitable tubular shape or may not extend all the way around the inlet valve. In some embodiments, a more general inner support could replace the inner flange and correspondingly the outer wall may not be annular if required so long as they are able to provide the functions of the inner flange and outer wall as described below.

As described above, the inlet valve 1151, inner flange 1155 and outer wall 1154 thereby form a top-hat profile of the distal sealing element 1150. This profile enables more secure fitment of the sealing element 1150 into the wetting mechanism as described below.

In this embodiment, an outer support in the form of an outer flange 1156 is attached to the outer edge of the outer wall 1154 and extends away from the outer wall 1154 in the same direction that the inner flange 1155 extends from the inlet valve 1151. In this embodiment, the outer flange 1156 has a length in the direction it extends away from the outer wall 1154 of 15-25% of the outer diameter of the outer wall 1154, for example 20% or about 60% longer than the inner flange 1155, for example about 3 mm. The outer flange 1156 is open-ended and frustoconical in shape and has an outer diameter at a distal end (distal from the outer wall 1154) approximately 1-5% larger than the outer diameter of the outer wall 1154, for example 2.5%.

In this embodiment, the outer flange 1156 comprises a sealing member in the form of a sealing rib 1157 arranged adjacent to the distal end. The sealing rib 1157 is a semi-circular protrusion from the outer surface of the outer flange 1156 that extends around the entire perimeter of the outer flange 1156. In this embodiment, the sealing rib 1157 does not protrude from an inner surface of the outer flange 1156, but in other embodiments it may do. The sealing rib 1157 has an outer diameter at its widest point approximately 13-17% larger than the outer diameter of the outer wall 1154, for example 15%. Thus, the sealing rib 1157 has a thickness approximately equal to 25% the thickness of the outer flange 1156. The sealing rib 1157 spans approximately 20-30% of the length of the outer flange 1154, for example 25%.

While not described in detail, the proximal sealing element 1130 is structurally identical to the distal sealing element 1150, as such, while only the distal sealing element 1150 is described above, this description also applies to the proximal sealing element 1130 which has corresponding features identified with corresponding numerals in the figures. The one exception being that the feature corresponding to the inlet valve 1151 of the proximal sealing element 1150 is called the outlet valve 1131 of the distal sealing element 1130 as the catheter 1200 enters the wetting mechanism 1100 through the inlet valve 1151 and exits through the outlet valve 1131 as described below.

In this embodiment the inserter tip 1120 comprises an insertion tube 1121 through which the proximal end 1201 of the catheter 1200 exits the wetting mechanism 1100 and passes into the user's body. The insertion tube 1121 is cylindrical with an inner diameter that is larger than the outer diameter of the catheter 1200 but less than the outer diameter of the inlet valve 1131 of the distal sealing element 1130, for example 7-9 mm or about 8 mm. The insertion tube 1121 has a constant thickness that is also the same as the wall thickness of the other parts of the inserter tip 1120 of about 1 mm. The insertion tube 1121 is therefore configured to be inserted into the urethra during use such that the catheter 1200 passes directly from the wetting mechanism 1100 into the user's body, this helps to reduce the risk of infection and discomfort as the catheter 1200 is guided smoothly into the body by the insertion tube 1121.

In this embodiment, the insertion tube 1121 is capped at one end with a hemicylindrical dome 1124 comprising two orthogonal slits 1122 defining four flaps 1123 in a similar arrangement to the inlet valve 1151 of the proximal sealing element 1150. The slits 1122 are configured to allow the flaps 1123 to separate as the proximal end 1201 of the catheter 1200 passes out through the dome 1124 from inside the insertion tube 1121. The domed shape also helps to facilitate comfortable insertion of the insertion tube 1121 into the body if required.

In this embodiment, the inserter tip 1120 comprises a skirt 1125 that extends away from the insertion tube 1121 at an end distal from the dome 1124. The skirt 1125 is planar and annular, with an inner diameter corresponding to the outer diameter of the insertion tube 1121 from which it extends and an outer diameter that is about two times larger than its inner diameter, for example 10-20 mm for example about 16 mm. In this embodiment, the insertion tube 1121 has a length from the tip of the dome 1124 to where it meets the skirt 1125 that corresponds to a safe insertion distance for the insertion tube 1121 within the body, for example 10-30 mm, or preferably 20 mm.

In this embodiment, the skirt 1125 provides a stop to prevent excessive insertion of the insertion tube 1121 into the body. To improve comfort in use, a proximal surface 1125a of the skirt 1125 is gently curved away from the insertion tube 1121 and the skirt 1125 therefore has a length measured parallel to the axis of the insertion tube 1121 of 2-4 mm, for example 2.3 mm.

In this embodiment, on an opposite distal surface 1125b of the skirt 1125 there is provided an inner sealing tube 1126 and an outer sealing tube 1127 arranged co-axially. Both tubes 1126, 1127 extend away from the skirt 1125 in a direction parallel to the insertion tube 1121 and terminate at a distance from the insertion tube 1121 and measured parallel to the insertion tube 1121 of 4-8 mm, for example about 6 mm. As both the inner sealing tube 1126 and outer sealing tube 1127 terminate the same distance from the insertion tube 1121, the inner sealing tube 1126 is slightly longer than the outer sealing tube 1127 on account of the curvature of the distal surface 1125b of the skirt 1125.

In this embodiment, the inner sealing tube 1126 is configured to receive the outlet valve 1131 and inner flange 1134 of the proximal sealing element 1130 within it to support the outlet valve 1131 in a radial direction, that is in directions perpendicular to the axial direction and parallel to the length of the inner sealing tube 1126. The inner sealing tube 1126 is therefore cylindrical with uncapped ends and has an inner diameter that is between the inner and outer diameters of the inner flange 1134, for example about 8.0-8.5 mm.

In this embodiment, the inner sealing tube 1126 and outer sealing tube 1127 are configured to abut the outer wall 1135 of the proximal sealing element 1130 to inhibit movement of the proximal sealing element 1130 towards the inserter tip 1110 in the axial direction. Thus, the outer sealing tube 1127 has a diameter that is substantially equal to the diameter of the outer flange 1136 and/or outer diameter of the outer wall 1135 such that the outer wall 1135 of the proximal sealing element 1130 can bear against the inner and outer sealing tubes 1126, 1127.

In this embodiment, the outer sealing tube 1127 is cylindrical with four gaps 1128 in the tube 1127 that extend from the free end of the tube 1127 in the axial direction approximately 60-70% of the way towards the distal surface 1125b. Each gap 1128 covers an arc length around circumference of the tube 1127 of 5-15 degrees, for example 10 degrees. Each gap 1128 has straight sides with a semi-circular end closest to the distal surface 1125b. The four gaps 1128 are spaced around the circumference of the tube 1127 with alternating angular separations of 65 degrees and then 115 degrees to define two opposing locking tongues 1129 that each cover an arc length of 65 degrees around the circumference of the tube 1127. At the free end of each locking tongue 1129, a locking protrusion 1129a extends out from the tube 1127 in the radial direction increasing the thickness of the tongue 1129 in this region by 40-60%, for example 50%. Each locking protrusion 1129a also covers approximately 25% of the length of each gap 1128 in the axial direction.

In this embodiment, each locking protrusion 1129a is configured to engage with a locking slot 1141 of the body 1140 described further below. The gaps 1128 provide a line of weakness in the tube 1127 that allows each locking protrusion 1129a to flex in the radial direction to ensure tight engagement with the locking slot 1141. To assist with flexing, each protrusion 1129a is wedge-shaped across two-thirds of its length. The effective thickness of the tube 1127 in a region of the locking protrusion 1129a is therefore a minimum at the free end of the tube 1127, then increases linearly to 50% more than the minimum thickness due to the wedge-shaped nature of the protrusion 1129a and is finally constant over the final third of the protrusion 1129a before extending radially inwards to meet the tube 1127.

In this embodiment, the base 1160 shares many of the same features as the inserter tip 1120 and so like numerals are used for like features and only the differences are described below.

In this embodiment, the base 1160 also comprises an insertion tube 1161, however, the insertion tube 1161 configured to guide the catheter 1200 from the sleeve into the wetting mechanism 1100 as described below and as such is open ended. The insertion tube 1161 is also 20% larger in diameter compared to the insertion tube 1121 of the inserter tip 1120 to make location of the proximal end 1201 of the catheter 1200 within the insertion tube 1161 of the base 1160 easier.

In this embodiment the body 1140 is cylindrical with open ends and an inner diameter that matches the outer diameter of the outer sealing tube 1127 of the inserter tip 1120 such that the outer sealing tube 1127 just fits within the body 1140. The body 1140 has an outer diameter that is 10-15% larger than its inner diameter, for example 12.5%, and equal to the outer diameter of the skirt 1125 (about 16 mm), 1165 to provide a smooth outer surface of the wetting mechanism 1100 when put together as described below. The body 1140 has a length perpendicular to its diameter and parallel to the axial direction that is 55-65% larger than its outer diameter, for example 60% or a length of about 25 mm.

In this embodiment, the body 1140 comprises four locking slots 1141 arranged in two pairs, one pair associated with the locking protrusions 1129a of the inserter tip 1120 and the other pair associated with the locking protrusions 1169a of the base 1160. Each slot 1141 is sized to receive a locking protrusion 1129a, 1169a and thus extends around the circumference of the body 1140 with an arc length around the circumference equivalent to 65 degrees. The two slots 1141 in each pair are separated by an angle of 115 degrees such that they align with the correct locking protrusions 1129a, 1169a. The slots 1141 are also separated from the open ends of the body 1140 by a distance equivalent to the separation between each locking protrusion 1129A, 1169a and the skirt 1125, 1165 respectively.

In this embodiment, at each end of the body 1140 the inner diameter of the body linearly increases over a distance of 2-3% of the length of the body 1140 to meet the outer diameter of the body 1140 and provide a wedge shaped profile around the circumference of each end of the body 1140 which assists with deformation of the locking protrusions 1129a, 1169a as the wetting mechanism 1100 is put together as described below.

In this embodiment, the body 1140 comprises eight ribs 1142 that extend in the axial direction along the body 1140. Each rib 1142 has a length in the axial direction equivalent to 60-70% of the length of the body 1140, for example 65% or about 16 mm. Each rib 1142 is arrange centrally with respect to the length of the body 1140 so that they occupy the middle 65% of the body 1140 thus leaving a space between the end of each rib 1142 and the inserter tip 1120 or base 1160 that is large enough for outer wall 1131, 1151 of the proximal/distal sealing element 1130, 1150 respectively.

In this embodiment, each rib 1142 extends from the inner surface of the body 1140 radially inward by at most 15-25% of the inner diameter of the body 1140, for example about 20%. Each rib 1142 extends radially inward furthest at the midpoint along the length of the rib 1142 and least at each end where it extends 10-15% less than at the middle. This improves the strength of the rib in mechanically supporting the body 1140 and wetting mechanism 1100.

In this embodiment, each rib 1142 is joined to the body 1140 along the majority of its length except for at each end where a sealing slot 1143 is present to receive the outer flange 1136, 1156 of the distal and proximal sealing elements 1130, 1150. Each sealing slot 1143 extends in an axial direction into the rib 1142 5-15% of the length of the rib 1142, for example about 10% such that the length of the sealing slot 1143 in the axial direction plus the distance between the corresponding end of the rib 1142 and the locking slot 1141 is equal to or preferably slightly less than the length of the outer flange 1136, 1156. This ensures that the inserter tip 1120 and base 1160 can press the outer flange 1136, 1156 into sealing engagement with the body 1140 via the sealing slot 1143, for example via the inner and outer sealing tubes. The sealing slots 1143 thus together form a slot that receives the outer flange 1136, 1156.

In this embodiment, each sealing slot 1143 has a width in the radial direction that is the same as the thickness of the outer flange 1136, 1156, but less than the thickness of the sealing rib 1137, 1157. In addition, the inner diameter of the body 1140 is slightly less than the widest diameter of the sealing rib 1137, 1157. Thus, when the sealing rib 1137, 1157 is forced into the respective sealing slot 1143 by the inserter tip 1120/base 1160, the proximal/distal sealing element 1130, 1150 deforms to provide a fluid tight seal between the it and the body 1140 to form a wetting chamber 1101 for the catheter 1200 as described below as described below.

In this embodiment, to assist with location of the outer flange 1136, 1156 in the sealing slots 1143, the mouth of each sealing slot 1143 is tapered on a side distal from the body 1140 to guide the outer flange 1136, 1156 into the respective sealing slot 1143.

In this embodiment, the ribs 1142 are attached to the inside of the body 1140 with equal angular separations around the circumference of the body 1140. The end of each rib 1142 is configured to abut the outer wall 1134, 1154 of the respective proximal or distal sealing element 1130, 1150 to inhibit movement of the proximal and distal sealing elements 1130, 1150 towards the centre of the body 1140. Thus, the end of each rib 1152 provides a stop which all together provide an axial stop at each end of the body 1140. As described above, the ribs 1142 each abut the respective outer wall 1134, 1154 at a distance from the centre of the outer wall 1134, 1154 that is between the respective inner sealing tube 1126, 1166 and outer sealing tube 1127, 1167 of the inserter tip 1120 and base 1160.

In this embodiment, the cap 1110 is configured to cover the inserter tip 1120 and protect it before use. Consequently, the cap 1110 is a shell with the same shape as the inserter tip 1120 but a larger size such that it can efficiently enclose it and like numerals are used to denote similar features.

In this embodiment, the cap 1110 comprises a cap tube 1111 which is cylindrical with an inner diameter that matches the outer diameter of the insertion tube 1121. The cap tube 1111 is capped at one end with a hemicylindrical dome 1114 to fit over the dome 1124 of the inserter tip 1120.

In this embodiment, the cap 1110 comprises a skirt cover 1115 extending out from the open end of the cap tube 1111 and configured to overlie the skirt 1125 of the inserter tip 1120. The skirt cover has an outer diameter that is just larger than the outer diameter of the body 1140.

In this embodiment, the cap 1110 comprises a cap flange 1117 that extends from the outer edge of the skirt cover 1115 in the axial direction to overlie the body 1140. The cap flange 1117 has a length such that when the cap 1110 is placed over the inserter tip 1120, the cap flange 1117 extends down 1-2 mm past the bottom of the outer tube 1127.

In this embodiment, at the free end of the cap flange 1117 two gripping protrusions 1119a are arranged on the inner surface of the cap flange 1117 in positions corresponding to the locking protrusions 1129a of the inserter tip 1120. Consequently, the two gripping protrusions 1119a each cover an arc length of 65 degrees and are separated by arcs of 115 degrees around the circumference of the cap 1110.

In this embodiment, each gripping protrusion 1119a has a semi-circular cross-section with a radius of 50% of the wall thickness of the cap flange 1117, consequently, in the region of the gripping protrusions 1119a, the wall thickness of the cap flange 1117 is up to 50% greater than in other parts of the cap flange 1117.

In this embodiment, the gripping protrusions 1119a are configured to engage the slots 1141 of the body 1140 in a similar manner to the locking protrusions 1129a however, the gripping protrusions 1119a are more easily disengaged from the slots 1141 by the user on account of their semi-circular profile as described below. Of course, in other embodiments, the gripping protrusions 1119a may be a different shape or size, or may be formed from multiple smaller protrusions and still function in the same way.

In this embodiment, the cap 1110 further comprises a pull ring 1112 attached to the dome 1114. The pull ring 1112 is configured to allow the user to grasp the cap 1110 and pull it off the wetting mechanism 1110 by disengaging the gripping protrusions 1119a from the slots 1141 as described below. Of course, in other embodiments, a different grippable feature may be used in place of the pull ring, such as a tab.

Referring to FIGS. 13-14 the wetting mechanism 1100 is constructed by arranging the cap 1110, inserter tip 1120 and distal sealing element 1130 co-axially on one side of the body 1140 and the base 1160 and proximal sealing element 1150 co-axially on the other side of the body 1140 and moving all parts of the wetting mechanism 1110 together in the axial direction as described below. Of course, this can be done in a variety of different ways and the method described below is purely exemplary.

In this embodiment, the proximal sealing element 1150 is fitted onto the base 1160. To do this, the inlet valve 1151 is aligned co-axially with the inner tube 1166 of the base 1160 and the proximal sealing element 1150 moved with respect to the base 1160 so that the inlet valve 1151 is received within the inner tube 1166. In this position, the inner tube 1166 supports the inlet valve 1151 in radial directions via engagement between the inner tube 1166 and the inner flange 1155 of the proximal sealing element 1150. In addition, over insertion of the inlet valve 1151 into the inner tube 1166 is prevented by the outer wall 1154 abutting the inner and outer tubes 1166, 1167.

In this embodiment, the distal sealing element 1150 is fitted onto the inserter tip 1150 in an identical manner to the fitting of the proximal sealing element 1150 to the base 1160.

In this embodiment, the body 1140 is then fitted onto the base 1160 trapping the proximal sealing element 1150 therebetween. The body 1140 is co-axially aligned with the base 1140 and rotated about its axis such that the locking slots 1141 are aligned with the locking protrusions 1169a of the base 1160. The locking slots 1141 and locking protrusions 1169a thereby forming two pairs of interlocking members of the wetting mechanism spaced apart around the circumference of the wetting mechanism. The body 1140 is then moved axially towards the base 1160 and as it moves, the locking protrusions 1169a contact the body 1140 causing deformation of the locking tongues 1169 radially inwardly until the locking protrusions 1169a are able to lock in the locking slots 1141 and ensure the body 1140 and base 1160 cannot move with respect to one another.

In this embodiment, simultaneous to the body 1140 fitting to the base 1160, the outer flange 1156 of the proximal sealing element 1150 is received in the sealing slots 1143 and the sealing rib 1157 is driven into engagement with the inner surface of the body 1140 by the sealing slots 1143 and outer tube 1167 deforming the proximal sealing element 1150. In addition, due to the size of the inner tube 1166 the inner flange 1155 seals tight against the inner tube 1166 and due to the size of the sealing slots 1143 and body 1140, the outer flange 1156 seals tight against the body 1140. Thus, the outer wall 1154 is also compressed in the radial direction which helps to drive the sealing engagement of the body 1140 and proximal sealing element 1150.

In this embodiment, the base 1160 and body 1140 are now sealed together and the body 1140 may be filled with a wetting fluid to be used to wet the surface of the catheter. In this embodiment, the wetting fluid is water and interacts with the hydrophilic surface of the catheter 1100 to render it lubricious. In other embodiments, other wetting fluids may be used and they may be polar (e.g. water-based) or non-polar (e.g. oil-based) depending on the catheter's surface properties. In this embodiment, 12 ml of wetting fluid is placed inside the body 1140, of course in other embodiments more or less wetting fluid may be required.

As the wetting fluid is typically a liquid, when filling the body 1140 care must be taken to ensure the body is oriented with the sealed end below the open end. In other embodiments, an alternative to free wetting fluid may be used, for example an applicator device loaded with wetting fluid may be placed into or integrated with the body and may be configured to release wetting fluid to wet the catheter.

In this embodiment, the inserter tip 1120 is then fitted to the other end of the body 1140 in the same manner than the base 1160 was. Once the inserter tip 1120 is fitted to the body 1140, a wetting chamber 1101 is defined by the body 1140, proximal sealing element 1130 and distal sealing element 1150 and the wetting fluid within the body 1140 is prevent from exiting the wetting chamber 1101 by the seals between the respective components and also the inlet and outlet valves 1151, 1131 being in their normally closed states and presenting a concave face towards the wetting chamber 1101. Consequently, the wetting chamber 1101 of this embodiment has a dual role as a wetting chamber in which the catheter is wetted and also a fluid reservoir in which wetting fluid is stored prior to wetting the catheter. In other embodiments, a separate fluid reservoir may be used which delivers wetting fluid to the wetting chamber when needed to wet the catheter.

In this embodiment, the cap 1110 is then fitted to the wetting mechanism 1100 by aligning it with the inserter tip 1120 and the moving it axially onto the inserter tip 1120 until the gripping protrusions 1119a engage with the locking slots 1141 of the body 1140 ensuring that the cap 1110 cannot be unintentionally removed. Consequently, the gripping protrusions 1119a also form interlocking members of the wetting mechanism.

In this embodiment, the catheter 1200 is then arranged with the proximal end 1201 of the catheter 1200 just inside the insertion tube 1161 of the base 1160. The sleeve 1300 is then arranged around the catheter 1200 and is attached to the outside of the insertion tube 1161 by any suitable means to form a fluid-tight seal between the sleeve 1300 and base 1160, for example a weld; mechanical seal; heat seal; pressure seal; adhesive; solvent bond; ultraviolet bond; ultrasonic weld; laser weld; impulse weld; or friction weld.

Of course, in other embodiments, the base may have been fitted to the cap instead of the body and then filled with wetting fluid while sealed to the inserter tip with a later step of sealing the base to the body to form the wetting chamber.

Thus, as the outlet valve 1131 is received by the inner sealing tube 1126 the inner flange 1134 contacts the inner sealing tube 1126 and the proximal sealing element 1130 is deformed in the radial direction towards the axis of the inner sealing tube 1126.

Referring to FIG. 1, in this embodiment, the catheter assembly 1000 is a closed catheter assembly and further comprises a fluid collection bag 1400 configured to receive fluid from the distal end 1202 of the catheter 1200. In this embodiment, the distal end 1202 comprises a funnel 1203 and the funnel 1203 is arranged within the fluid collection bag 1400. A fluid-tight seal is provided between the funnel 1203 and fluid collection bag 1400 to prevent leaks of fluid. A fluid-tight seal is also provided between the sleeve 1300 and funnel 1203 to ensure fluid cannot leak out of the sleeve 1300.

In this embodiment, the funnel 1203 comprises bypass tubes 1203 configured to allow liquid inside the sleeve 1300 to pass into the fluid collection bag 1400.

Referring to FIGS. 14 to 17, in this embodiment, to prepare the catheter 1200 for use, the user grasps the catheter 1200 via the sleeve 1300 and progressively moves the proximal end 1201 of the catheter 1200 into the inlet valve 1151. The flaps 1153 separate opening the inlet valve 1151 and allowing the catheter 1200 to enter the wetting chamber 1101 to be wetted by wetting fluid Advantageously, this ensures the first part of the catheter 1200 to enter the body, that is the proximal end 1201, is the part most likely to be wetted by the wetting mechanism 1100. This helps reduce the likelihood of discomfort or injury during use.

In this embodiment, opening of the inlet valve 1151 also allows wetting fluid into the sleeve 1300 to wet the rest of the catheter 1200. This wetting fluid may pass into the fluid collection bag 1400 via the funnel 1203.

In this embodiment, the catheter 1200 is moved through the wetting chamber 1101 until the proximal end 1201 contacts the outlet valve 1131 and moves through the outlet valve 1131 by separating the flaps 1133 and opening the outlet valve 1131.

In this embodiment, the cap 1110 is then removed from the wetting mechanism 1100 by grasping the wetting mechanism 1100 in one hand and then pulling the cap off the wetting mechanism 1100 to disengage the gripping protrusions 1119a from the locking slots 1141.

In this embodiment, the inserter tip 1120 is then inserted into the urethra and the catheter 1200 is moved through the insertion tube 1121 until it passes out of the wetting mechanism 1100 and into the body by separating the flaps 1113 at the tip of the inserter tip 1110. The catheter 1200 may then be inserted into the body until urine flows through the catheter 1200 and into the fluid collection bag 1400.

Referring to FIG. 18, a second embodiment of a catheter assembly 3000 shares many of the same features as the first embodiment. Consequently, like numerals are used to denote like features and the only difference between the two embodiments is that in the second embodiment the catheter assembly 3000 does not comprise a fluid collection bag. Thus, fluid may flow directly out of the funnel to be disposed off, for example into a toilet. The catheter assembly 3000 is therefore an “open” catheter assembly.

The one or more embodiments are described above by way of example only. Many variations are possible without departing from the scope of protection afforded by the appended claims.

1. A catheter assembly comprising: a catheter comprising a proximal end for insertion into the body and a distal end, and a wetting mechanism arranged at the proximal end of the catheter, wherein the wetting mechanism comprises a body, the body defines a wetting chamber through which the catheter may be moved to wet the catheter, and the wetting mechanism comprises a slit valve configured to allow passage of the catheter into the wetting chamber and to inhibit release of fluid from the wetting chamber.

2. A catheter assembly according to claim 1 or claim 2 wherein the catheter is formed of a hydrophilic material and the wetting chamber is configured to contain a wetting liquid, wherein the slit valve is configured to inhibit the release of the wetting liquid from the wetting chamber.

3. A catheter assembly according to any preceding claim wherein the slit valve comprises at least one slit defining at least two flaps that are separatable to allow passage of the catheter through the slit valve.

4. A catheter assembly according to any preceding claim wherein the wetting mechanism comprises two slit valves: an inlet valve and an outlet valve, the inlet valve configured to allow passage of the catheter into the wetting chamber and to inhibit release of fluid from the wetting chamber, and the outlet valve configured to allow passage of the catheter out of the wetting chamber and inhibit release of fluid from the wetting chamber.

5. A catheter assembly according to claim 5 wherein each of the inlet and outlet valve have a curved surface and the inlet and outlet valve are arranged in the wetting mechanism with opposing curvature.

6. A catheter assembly according to claim 5 or 6 further comprising an inserter tip configured to engage one end of the body with the outlet valve therebetween and is shaped to aid insertion of the catheter into the body.

7. A catheter assembly according to claim 7 wherein the inserter tip comprises an insertion tube capped with a dome, the dome comprising at least one slit defining at least two flaps, the at least two flaps configured to separate to allow the catheter to pass through the dome.

8. A catheter assembly according to claim 7 or 8 further comprising a cap, the cap provided over and around the inserter tip.

9. A catheter assembly according to any preceding claim wherein the slit valve has a curved surface.

10. A catheter assembly according to claim 10 wherein the slit valve is domed.

11. A catheter assembly according to claim 11 wherein the slit valve has a dome height of no more than 30% the diameter of the slit valve.

12. A catheter assembly according to any of claims 10 to 12 wherein the slit valve is convex on a side facing away from the wetting chamber.

13. A catheter assembly according to any preceding claim wherein the slit valve is normally closed.

14. A catheter assembly according to any preceding claim wherein the slit valve is formed of a flexible resilient material.

15. A catheter assembly according to any preceding claim wherein the wetting mechanism comprises a sealing element, the sealing element comprising the slit valve, wherein the sealing element has a stepped profile.

16. A catheter assembly according to any preceding claim wherein the wetting mechanism is tubular and comprises a sealing element, body and a base, the base comprising an opening sized to allow the catheter to pass through the base and into the wetting chamber, the sealing element comprising the slit valve forming an inlet valve, and the sealing element comprising a material that is more flexible than the body and the base, and wherein the sealing element is resiliently deformed by the body and base to provide a fluid-tight seal between the body and the base

17. A catheter assembly comprising: a catheter comprising a proximal end for insertion into the body and a distal end, and a wetting mechanism arranged at the proximal end of the catheter, wherein the wetting mechanism comprises a body, a base and a sealing element, the body defining a tubular wetting chamber through which the catheter may be moved to wet the catheter, the base comprising an opening sized to allow the catheter to pass through the base and into the wetting chamber, the sealing element comprising an inlet valve configured to allow passage of the catheter into the wetting chamber and inhibit release of fluid from the wetting chamber, the sealing element comprising a material that is more flexible than the body and the base, and wherein the sealing element is resiliently deformed by the body and base to provide a fluid-tight seal between the body and the base.

18. A catheter assembly according to claim 16 or 17 wherein the sealing element has a stepped profile.

19. A catheter assembly according to claim 18 wherein the sealing element has a top-hat profile.

20. A catheter assembly according to any of claims 16 to 19 wherein at least part of the sealing element is compressed in the axial direction by the base and/or body, the axial direction defined as the direction in which the catheter passes into the wetting chamber through the inlet valve.

21. A catheter assembly according to any of claims 16 to 22 wherein the sealing element comprises an outer flange extending in the axial direction and being compressed by the base and body.

22. A catheter assembly according to claim 21 wherein the body comprises a slot and the outer flange is received in and resiliently deformed by the slot.

23. A catheter assembly according to claim 21 or 22 wherein the outer flange is compressed in the axial direction between the base and body.

24. A catheter assembly according to any of claims 16 to 23 wherein at least part of the sealing element is compressed in a radial direction perpendicular to the axial direction by the base and body.

25. A catheter assembly according to claim 24 wherein the sealing element comprises an inner flange and an outer flange both extending in the axial direction and separated by an outer wall, the body comprises a slot configured to receive the outer flange and the base comprises an inner sealing tube configured to abut the inner flange, wherein the distance measured in the radial direction between an inner diameter of the slot and an inner diameter of the inner sealing tube is greater than the distance between an outer diameter of the inner flange and an inner diameter of the outer flange.

26. A catheter assembly according to any of claims 16 to 25 wherein the wetting mechanism comprises two or more interlocking members configured to secure the base and body together.

27. A catheter assembly according to claim 26 comprising two or more pairs of interlocking members.

28. A catheter assembly according to claim 27 wherein the two or more pairs of interlocking members are spaced apart around the circumference of the wetting mechanism.

29. A catheter assembly according to any of claims 26 to 28 wherein at least two interlocking members comprise at least one locking protrusion and at least one locking slot.

30. A catheter assembly as claimed in any preceding claim wherein the body is tubular and each end of the body is configured to receive a sealing element.

31. A catheter assembly as claimed in claim 30 wherein the wetting mechanism comprises two sealing elements and two bases to seal both ends of the body.

32. A catheter assembly according to any preceding claim wherein the wetting mechanism comprises a sealing element comprising an inlet valve configured to allow passage of the catheter into the wetting chamber in an axial direction to be wetted by wetting fluid, the inlet valve defines a transverse plane perpendicular to the axial direction, the sealing element comprises: a stepped profile, an outer wall extending parallel to the transverse plane but offset from the inlet valve, an inner support extending in the axial direction and connecting the outer wall to the inlet valve, wherein the wetting mechanism abuts the inner support to inhibit movement of the inlet valve in the transverse plane, and the wetting mechanism abuts the outer wall to inhibit movement of the inlet valve in the axial direction.

33. A catheter assembly comprising: a catheter comprising a proximal end for insertion into the body and a distal end, and a wetting mechanism arranged at the proximal end of the catheter; wherein the wetting mechanism comprises a wetting chamber and a sealing element, the sealing element comprising an inlet valve configured to allow passage of the catheter into the wetting chamber in an axial direction to be wetted by wetting fluid, the inlet valve is configured to inhibit release of fluid from the wetting chamber, the inlet valve defines a transverse plane perpendicular to the axial direction, the sealing element comprises: a stepped profile, an outer wall extending parallel to the transverse plane but offset from the inlet valve, an inner support extending in the axial direction and connecting the outer wall to the inlet valve, wherein the wetting mechanism abuts the inner support to inhibit movement of the inlet valve in the transverse plane, and the wetting mechanism abuts the outer wall to inhibit movement of the inlet valve in the axial direction.

34. A catheter assembly according to any preceding claim wherein the catheter is a urinary catheter.

35. A catheter assembly according to claim 35 wherein the catheter is an intermittent male urinary catheter.

36. A catheter assembly according to any preceding claim wherein the catheter comprises a hydrophilic material and the wetting chamber is configured to contain a wetting liquid.

37. A catheter assembly according to any of claims 32 to 36 wherein the inner support is attached to an outer edge of the inlet valve.

38. A catheter assembly according to any of claims 32 to 37 wherein the inner support comprises an inner flange surrounding the inlet valve in the transverse plane and the inner flange is frustoconical.

39. A catheter assembly according to any of claims 32 to 38 wherein the wetting mechanism comprises a transverse support structure configured to abut the inner support and surround at least part of the inner support.

40. A catheter assembly according to claim 39 wherein the transverse support structure may surround the inner support in the transverse plane.

41. A catheter assembly according to claim 39 or 40 wherein the transverse support structure comprises an inner sealing tube and the inner sealing tube is cylindrical.

42. A catheter assembly according to any of claims 32 to 41 wherein the outer wall surrounds the inlet valve in the transverse plane.

43. A catheter assembly according to claim 42 wherein the outer wall comprises an inner perimeter corresponding to an outer perimeter of the inlet valve.

44. A catheter assembly according to claim 43 wherein the inner perimeter of the outer wall is spaced outside the outer perimeter of the inlet valve in the transverse direction.

45. A catheter assembly according to any of claims 32 to 44 further comprising an axial support structure configured to abut the outer wall at two or more independent locations.

46. A catheter assembly according to any of claims 32 to 45 wherein the outer wall is annular and outer wall is compressed in a direction parallel to the transverse plane by the wetting mechanism.

47. A catheter assembly according to any of claims 32 to 46 the outer wall further comprising an outer flange, the outer flange being compressed by the wetting mechanism.

48. A catheter assembly according to claim 47 wherein the wetting mechanism further comprising a slot configured to receive the outer flange.

49. A method of manufacturing a catheter assembly comprising providing a catheter comprising a proximal end for insertion into the user and a distal end and a wetting mechanism, wherein the wetting mechanism comprises a body, the body defines a wetting chamber through which the catheter may be moved to wet the catheter, and wetting mechanism comprises a slit valve configured to allow passage of the catheter into the wetting chamber and inhibit release of fluid from the wetting chamber, the method comprising arranging the wetting mechanism at the proximal end of the catheter.

50. A method of manufacturing a catheter assembly comprising providing a catheter comprising a proximal end for insertion into the body and a distal end and a wetting mechanism wherein the wetting mechanism comprises a body, a base and a sealing element comprising an inlet valve configured to allow passage of the catheter into the wetting chamber and inhibit release of fluid from the wetting chamber, the body defining a tubular wetting chamber through which the catheter may be moved to wet the catheter, the base comprising an opening sized to allow the catheter to pass through the base and into the wetting chamber, the sealing element comprising a material that is more flexible than the body and the base, and wherein the sealing element is resiliently deformed by the body and base to provide a fluid-tight seal between the body and the base, the method comprising arranging the wetting mechanism at the proximal end of the catheter.

51. A method as claimed in claim 49 or 50 wherein the body is tubular and the method further comprising sealing both ends of the body with two sealing elements and two bases.

52. A method of manufacturing a catheter assembly comprising providing a catheter comprising a proximal end for insertion into the body and a distal end and a wetting mechanism, wherein the wetting mechanism comprises a wetting chamber and a sealing element, the sealing element comprising an inlet valve configured to allow passage of the catheter into the wetting chamber in an axial direction to be wetted by wetting fluid, the inlet valve is configured to inhibit release of fluid from the wetting chamber, the inlet valve defines a transverse plane perpendicular to the axial direction, the sealing element comprising a stepped profile, the sealing element comprising an outer wall extending parallel to the transverse plane but offset from the inlet valve, the sealing element comprising an inner support extending in the axial direction and connecting the outer wall to the inlet valve, the wetting mechanism abuts the inner support to inhibit movement of the inlet valve in the transverse plane, and the wetting mechanism abuts the outer wall to inhibit movement of the inlet valve in the axial direction, the method comprising arranging the wetting mechanism at the proximal end of the catheter.

53. A method of wetting a catheter, the catheter comprising a proximal end for insertion into the body and a distal end, the method comprising providing a wetting mechanism at the proximal end of the catheter, wherein the wetting mechanism comprises a slit valve and a body defining a wetting chamber comprising wetting fluid, the slit valve configured to inhibit release of wetting fluid from the wetting chamber, the method comprising moving the catheter through the slit valve and into the wetting chamber to wet the catheter.

54. A method according to claim 53 wherein the slit valve comprises at least two flaps and the method comprises separating the flaps with the catheter.

55. A method of wetting a catheter comprising a proximal end for insertion into the body and a distal end, the method comprising providing a wetting mechanism at the proximal end of the catheter, wherein the wetting mechanism comprises a body, a base and a sealing element, the method comprising moving the catheter through an opening in the base, through an inlet valve in the sealing element and into a tubular wetting chamber defined by the body, wherein the inlet valve is configured to inhibit release of fluid from the wetting chamber, the sealing element comprises a material that is more flexible than the body and the base, and wherein the sealing element is resiliently deformed by the body and base to provide a fluid-tight seal between the body and the base.

56. A method of wetting a catheter comprising a proximal end for insertion into the body, the method comprising arranging a wetting mechanism at the proximal end of the catheter, wherein the wetting mechanism comprises a wetting chamber and a sealing element, the method comprising moving the catheter in an axial direction through an inlet valve in the sealing element and into the wetting chamber, wherein, the inlet valve is configured to inhibit release of fluid from the wetting chamber, the inlet valve defines a transverse plane perpendicular to the axial direction, the sealing element comprises a stepped profile, the sealing element comprises an outer wall extending parallel to the transverse plane but offset from the inlet valve, and an inner support extending in the axial direction and connecting the outer wall to the inlet valve, wherein the wetting mechanism abuts the inner support to inhibit movement of the inlet valve in the transverse plane, and the wetting mechanism abuts the outer wall to inhibit movement of the inlet valve in the axial direction.

57. A method according to any of claims 49 to 56 using the catheter assembly of any of claims 1 to 48.
